(19) United States
(12) Patent Application Publication
(10) Pub. No.: US 2019/0185930 A1
(43) Pub. Date: Jun. 20, 2019

(54) METHODS OF PREPARING A SEQUENCING LIBRARY ENRICHED FOR DUPLEX DNA MOLECULES

(71) Applicant: GRAIL, Inc., Menlo Park, CA (US)

(72) Inventors: Lijuan Ji, Santa Clara, CA (US); Nathan Hunkapiller, Belmont, CA (US); Nicholas Eattock, Fremont, CA (US); Byoungsok Jung, Atherton, CA (US)

(21) Appl. No.: 16/228,466

(22) Filed: Dec. 20, 2018

Related U.S. Application Data
(60) Provisional application No. 62/608,538, filed on Dec. 20, 2017.

(51) Int. Cl. C12Q 1/6874

(52) U.S. Cl. CPC C12Q 1/6874

(57) ABSTRACT

Methods for preparing sequencing libraries from a DNA-containing test sample, as well as methods for correcting sequencing-derived errors in sequence reads, and methods for identifying rare variants in a test sample, are provided.

### CROSS-REFERENCE TO RELATED APPLICATIONS

Under 35 U.S.C. § 119(e), this application claims priority benefit of the filing date of U.S. Provisional Patent Application No. 62/608,538, filed on Dec. 20, 2017, the disclosure of which application is herein incorporated by reference in its entirety.

### FIELD OF THE INVENTION

The present invention relates to molecular biology techniques and methods for preparing sequencing libraries from a DNA-containing test sample, as well as methods for correcting sequencing-derived errors in sequence reads, and methods for identifying rare variants in a test sample.

### BACKGROUND OF THE INVENTION

Analysis of circulating cell-free DNA (cfDNA) using next generation sequencing (NGS) is recognized as a valuable tool for detection and diagnosis of cancer. Identifying rare variants indicative of cancer using NGS often requires deep sequencing of circulating cfDNA from a patient test sample. Alternatively, many tumor-derived variants can also be identified using less expensive, lower depth, whole exome sequencing approaches. However, errors introduced during sample preparation and sequencing can make accurate identification of variants difficult.

Duplexed sequence reads are critical for error correction in sequencing applications that typically use low input levels of material and/or have limited sequencing coverage (e.g., analysis of cfDNA). For error correction, particularly in limited depth exome sequencing, it is important to avoid sequencing non-duplex DNA molecules. Current protocols for preparing a sequencing library from double-stranded DNA typically includes DNA end repair, 3′ end A-tailing, ligation of sequencing adapters to the double-stranded (duplexed) DNA, and polymerase chain reaction (PCR) amplification to enrich for adapter-ligated DNA molecules. The procedure requires four successful ligation events to obtain sequenceable fragments for both the forward and reverse strands of a double-stranded DNA molecule. If a single ligation event fails during library preparation, one strand of the duplexed library fragment will not be amplified and a non-duplexed read will be observed during sequence analysis. However, as one of skill in the art would readily recognize, these individual ligations events are not 100% efficient, and sequence information from the test sample can be lost. Accordingly, there is a need in the art for new methods of preparing a sequencing library that enrich for duplexed DNA molecules, thereby increasing duplex reads in sequencing.

### SUMMARY OF THE INVENTION

Aspects of the invention include methods for preparing a sequencing library, the methods comprising: (a) obtaining a test sample comprising a plurality of double-stranded DNA (dsDNA) molecules having first and second ends, wherein the dsDNA molecules comprise a forward strand sequence and a reverse complement strand sequence; (b) providing a plurality of loop-shaped double-stranded DNA (dsDNA) adapters, wherein the loop-shaped dsDNA adapters comprise a recognition site for nuclease digestion; (c) modifying the plurality of dsDNA molecules for adapter ligation; (d) ligating the loop-shaped dsDNA adapters to both ends of the plurality of dsDNA molecules, to generate a plurality of circular adapter-dsDNA-adapter constructs; (e) amplifying the plurality of circular adapter-dsDNA-adapter constructs to generate a plurality of concatemer amplicons comprising alternating forward and reverse complement strands originating from the dsDNA molecules; and (f) digesting the plurality of concatemer amplicons to generate a plurality of single-stranded DNA molecules comprising the forward and the reverse complement strand sequences, thereby generating a sequencing library. In some embodiments, the loop-shaped dsDNA adapters comprise a unique molecular identifier (UMI).

In some embodiments, the methods further comprise: (g) sequencing at least a portion of the sequencing library to obtain a plurality of sequence reads; (h) grouping the sequence reads into families based on the UMIs, wherein the families comprise a first set of forward strand sequences, each having a first UMI, and a second set of reverse complement strand sequences, each having a second UMI, wherein the second UMI sequence is complementary to the first UMI sequence; and (i) comparing the sequence reads within each family to generate a consensus sequence for each of the families. In some embodiments, the methods further comprise: (j) aligning the one or more consensus sequences to a reference sequence and identifying the one or more consensus sequences as one or more rare variants if the one or more consensus sequences vary from the reference sequence at one or more nucleotide positions.

Aspects of the invention include methods for preparing a sequencing library, the methods comprising: (a) obtaining a test sample comprising a plurality of double-stranded DNA (dsDNA) molecules having first and second ends, wherein the dsDNA molecules comprise a forward strand sequence and a reverse complement strand sequence; (b) providing a plurality of loop-shaped double-stranded DNA (dsDNA) adapters, wherein the loop-shaped dsDNA adapters comprise a recognition site for nuclease digestion; (c) modifying the plurality of dsDNA molecules for adapter ligation; (d) ligating the loop-shaped dsDNA adapters to both ends of the plurality of dsDNA molecules, to generate a plurality of circular adapter-dsDNA-adapter constructs; (e) digesting unligated nucleic acids with an exonuclease; (f) cleaving the plurality of loop-shaped dsDNA adapters at the recognition site with a nuclease to generate a sequencing library. In some embodiments, the loop-shaped dsDNA adapters comprise a unique molecular identifier (UMI).

In some embodiments, the methods further comprise: (g) sequencing at least a portion of the sequencing library to obtain a plurality of sequence reads; (h) grouping the sequence reads into families based on the UMIs, wherein the families comprise a first set of forward strand sequences, each having a first UMI, and a second set of reverse complement strand sequences, each having a second UMI, wherein the second UMI sequence is complementary to the first UMI sequence; and (i) comparing the sequence reads within each family to generate a consensus sequence for each of the families. In some embodiments, the methods further comprise: (j) aligning the one or more consensus sequences to a reference sequence and identifying the one or more consensus sequences as one or more rare variants if the one or more consensus sequences vary from the reference sequence at one or more nucleotide positions.

Aspects of the invention include methods for preparing a sequencing library, the methods comprising: (a) obtaining a test sample comprising a plurality of double-stranded DNA (dsDNA) molecules having first and second ends, wherein the dsDNA molecules comprise a forward strand sequence and a reverse complement strand sequence; (b) providing a plurality of loop-shaped double-stranded DNA (dsDNA) adapters, wherein the loop-shaped dsDNA adapters comprise a recognition site for nuclease digestion; (c) modifying the plurality of dsDNA molecules for adapter ligation; (d) ligating the loop-shaped dsDNA adapters to both ends of the plurality of dsDNA molecules, to generate a plurality of circular adapter-dsDNA-adapter constructs; (e) digesting unligated DNA molecules with an exonuclease; (f) amplifying the plurality of circular adapter-dsDNA-adapter constructs to generate a plurality of concatemer amplicons comprising alternating forward and reverse complement strands originating from the dsDNA molecules; and (g) cleaving the plurality of loop-shaped dsDNA adapters at the nuclease recognition site to generate a plurality of single-stranded DNA molecules comprising the forward and the reverse complement strand sequences, thereby generating a sequencing library. In some embodiments, the loop-shaped dsDNA adapters comprise a unique molecular identifier (UMI).

In some embodiments, the methods further comprise: (h) sequencing at least a portion of the sequencing library to obtain a plurality of sequence reads; (i) grouping the sequence reads into families based on the UMIs, wherein the families comprise a first set of forward strand sequences, each having a first UMI, and a second set of reverse complement strand sequences, each having a second UMI, wherein the second UMI sequence is complementary to the first UMI sequence; and (j) comparing the sequence reads within each family to generate a consensus sequence for each of the families. In some embodiments, the methods further comprise: (k) aligning the one or more consensus sequences to a reference sequence and identifying the one or more consensus sequences as one or more rare variants if the one or more consensus sequences vary from the reference sequence at one or more nucleotide positions.

In some embodiments, the methods further comprise contacting the circular adapter-dsDNA-adapter constructs with a topoisomerase enzyme. In some embodiments, the dsDNA molecules are cell-free DNA (cfDNA) molecules. In some embodiments, the cfDNA molecules originate from healthy cells and from cancer cells. In some embodiments, the test sample is from whole blood, a blood fraction, plasma, serum, urine, fecal matter, saliva, a tissue biopsy, pleural fluid, pericardial fluid, cerebrospinal fluid (CSF), or peritoneal fluid. In some embodiments, modification of the plurality of dsDNA molecules comprises end-repairing and A-tailing prior to the ligation step. In some embodiments, the adapters further comprise a sample-specific index sequence. In some embodiments, the adapters further comprise a universal priming site. In some embodiments, the adapters further comprise one or more sequencing oligonucleotides for use in cluster generation and/or sequencing.

In some embodiments, the consensus sequence comprises a sequence of nucleotide bases, wherein each base is identified at a given position in the sequence when a specific base is present in a majority of the sequence reads of the family. In some embodiments, the consensus sequence comprises a sequence of nucleotide bases, wherein each base is identified at a given position in the sequence when a specific base is present in at least 70%, 80%, 90%, or 95% of the sequence reads comprising the family.

In some embodiments, the methods further comprise loading at least a portion of the sequence library into a sequencing flow cell and generating a plurality of sequencing clusters on the flow cell, wherein the clusters comprise the forward strand sequence and the reverse complement strand sequence. In some embodiments, the sequence reads are obtained from next-generation sequencing (NGS). In some embodiments, the sequence reads are obtained from massively parallel sequencing using sequencing-by-synthesis. In some embodiments, the sequence reads are obtained from paired-end sequencing. In some embodiments, the sequence reads comprise a read pair, wherein each read pair comprises a first read of the forward strand sequence and second read of the reverse complement strand sequence.

In some embodiments, the methods further comprise using the one or more rare variants to detect the presence or absence of cancer, determine cancer status, monitor cancer progression, and/or determine a cancer classification. In some embodiments, monitoring cancer progression further comprises monitoring disease progression, monitoring therapy, or monitoring cancer growth. In some embodiments, determining the cancer classification further comprises determining a cancer type and/or a cancer tissue of origin. In some embodiments, the cancer comprises a carcinoma, a sarcoma, a myeloma, a leukemia, a lymphoma, a blastoma, a germ cell tumor, or any combination thereof.

### BRIEF DESCRIPTION OF THE DRAWINGS

FIG. 1 is a flow diagram illustrating a method of preparing a sequencing library enriched for duplexed DNA, in accordance with one embodiment of the present invention;

FIG. 2 is a flow diagram illustrating a method of preparing a sequencing library enriched for duplexed DNA, in accordance with another embodiment of the present invention;

FIG. 3 is a flow diagram illustrating a method of preparing a sequencing library enriched for duplexed DNA, in accordance with still another embodiment of the present invention;

FIG. 4 is a flow diagram illustrating a method for preparing a sequencing library enriched for duplex DNA, in accordance with another embodiment;

FIG. 5 is a schematic diagram illustrating a loop adapter for amplification and enrichment of adapter-ligated duplexed DNA;

FIGS. 6A and 6B is a schematic showing pictorially some of the steps of the method of FIG. 4;

FIG. 7 is a flow diagram illustrating a method for error correction using a sequencing library prepared in accordance with the method of FIG. 1; and

FIG. 8 is a flow diagram illustrating a method for variant detection, using a sequencing library prepared in accordance with the method of FIG. 1.

FIG. 9, Panel A is a graph showing the number of collapsed reads based on the presence or absence of an exonuclease enzyme in the preparation protocol. Panel B is a graph showing the percentage of duplex DNA based on the presence or absence of an exonuclease enzyme in the preparation protocol.

### DEFINITIONS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit, unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application, as do the following, each of which is incorporated by reference herein in its entirety: Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6^{th} edition (Saunders, 2007).

All publications mentioned herein are expressly incorporated herein by reference to disclose and describe the methods and/or materials in connection with which the publications are cited.

The term “amplicon” as used herein means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references, each of which are incorporated herein by reference herein in their entirety: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g., “real-time PCR”, or “real-time NASBA” as described in Leone et al, Nucleic Acids Research, 26: 2150-2155 (1998), and like references.

As used herein, the term “amplifying” means performing an amplification reaction. A “reaction mixture” means a solution containing all the necessary reactants for performing a reaction, which may include, but is not be limited to, buffering agents to maintain pH at a selected level during a reaction, salts, co-factors, scavengers, and the like.

The terms “fragment” or “segment”, as used interchangeably herein, refer to a portion of a larger polynucleotide molecule. A polynucleotide, for example, can be broken up, or fragmented into, a plurality of segments, either through natural processes, as is the case with, e.g., cfDNA fragments that can naturally occur within a biological sample, or through in vitro manipulation. Various methods of fragmenting nucleic acids are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave a polynucleotide at known or unknown locations. Physical fragmentation methods may involve subjecting a polynucleotide to a high shear rate. High shear rates may be produced, for example, by moving DNA through a chamber or channel with pits or spikes, or forcing a DNA sample through a restricted size flow passage, e.g., an aperture having a cross sectional dimension in the micron or submicron range. Other physical methods include sonication and nebulization. Combinations of physical and chemical fragmentation methods may likewise be employed, such as fragmentation by heat and ion-mediated hydrolysis. See, e.g., Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range.

The terms “polymerase chain reaction” or “PCR”, as used interchangeably herein, mean a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors that are well-known to those of ordinary skill in the art, e.g., exemplified by the following references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including, but not limited to, RT-PCR, real-time PCR, nested PCR, quantitative PCR, multiplexed PCR, and the like. The particular format of PCR being employed is discernible by one skilled in the art from the context of an application. Reaction volumes can range from a few hundred nanoliters, e.g., 200 nL, to a few hundred μL, e.g., 200 μL. “Reverse transcription PCR,” or “RT-PCR,” means a PCR that is preceded by a reverse transcription reaction that converts a target RNA to a complementary single stranded DNA, which is then amplified, an example of which is described in Tecott et al, U.S. Pat. No. 5,168,038, the disclosure of which is incorporated herein by reference in its entirety. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); the disclosures of which are hereby incorporated by reference herein in their entireties. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out in the same reaction mixture, e.g., Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references, which are incorporated by reference herein in their entireties: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); and Becker-Andre et al, Nucleic Acids Research, 17: 9437-9446 (1989).

The term “primer” as used herein means an oligonucleotide, either natural or synthetic, that is capable, upon forming a duplex with a polynucleotide template, of acting as a point of initiation of nucleic acid synthesis and being extended from its 3′-end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic acid amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following reference that is incorporated by reference herein in its entirety: Dieffenbach, editor, PCR Primer: A Laboratory Manual, 2^{nd} Edition (Cold Spring Harbor Press, New York, 2003).

The terms “unique sequence tag”, “sequence tag”, “tag”, “unique molecular identifier”, “UMI”, or “barcode”, as used interchangeably herein, refer to an oligonucleotide that is attached to a polynucleotide or template molecule and is used to identify and/or track the polynucleotide or template in a reaction or a series of reactions. A sequence tag may be attached to the 3′- or 5′-end of a polynucleotide or template, or it may be inserted into the interior of such polynucleotide or template to form a linear conjugate, sometimes referred to herein as a “tagged polynucleotide,” or “tagged template,” or the like. Sequence tags may vary widely in size and compositions; the following references, which are incorporated herein by reference in their entireties, provide guidance for selecting sets of sequence tags appropriate for particular embodiments: Brenner, U.S. Pat. No. 5,635,400; Brenner and Macevicz, U.S. Pat. No. 7,537,897; Brenner et al, Proc. Natl. Acad. Sci., 97: 1665-1670 (2000); Church et al, European patent publication 0 303 459; Shoemaker et al, Nature Genetics, 14: 450-456 (1996); Morris et al, European patent publication 0799897A1; Wallace, U.S. Pat. No. 5,981,179; and the like. Lengths and compositions of sequence tags can vary widely, and the selection of particular lengths and/or compositions depends on several factors including, without limitation, how tags are used to generate a readout, e.g., via a hybridization reaction or via an enzymatic reaction, such as sequencing; whether they are labeled, e.g., with a fluorescent dye or the like; the number of distinguishable oligonucleotide tags required to unambiguously identify a set of polynucleotides, and the like, and how different the tags of a particular set must be in order to ensure reliable identification, e.g., freedom from cross hybridization or misidentification from sequencing errors. In one aspect, sequence tags can each have a length within a range of from about 2 to about 36 nucleotides, or from about 4 to about 30 nucleotides, or from about 4 to about 20 nucleotides, or from about 8 to about 20 nucleotides, or from about 6 to about 10 nucleotides. In one aspect, sets of sequence tags are used, wherein each sequence tag of a set has a unique nucleotide sequence that differs from that of every other tag of the same set by at least two bases; in another aspect, sets of sequence tags are used wherein the sequence of each tag of a set differs from that of every other tag of the same set by at least three bases.

The term “enrich” as used herein means to increase a proportion of one or more target nucleic acids in a sample. An “enriched” sample or sequencing library is therefore a sample or sequencing library in which a proportion of one of more target nucleic acids has been increased with respect to non-target nucleic acids in the sample.

The term “deplete” as used herein means to decrease a proportion of one or more target nucleic acids in a sample. A “depleted” sample or sequencing library is therefore a sample or sequencing library in which a proportion of one of more target nucleic acids has been decreased with respect to non-target nucleic acids in the sample.

The terms “subject” and “patient” are used interchangeably herein and refer to a human or non-human animal who is known to have, or potentially has, a medical condition or disorder, such as, e.g., a cancer.

The term “sequence read” as used herein refers to nucleotide sequences read from a sample obtained from a subject. Sequence reads can be obtained through various methods known in the art.

The terms “circulating tumor DNA” or “ctDNA” and “circulating tumor RNA” or “ctRNA” refer to nucleic acid fragments (DNA or RNA) that originate from tumor cells or other types of cancer cells, which may be released into a subject's bloodstream as a result of biological processes, such as apoptosis or necrosis of dying cells, or may be actively released by viable tumor cells.

### DETAILED DESCRIPTION OF THE INVENTION

Aspects of the invention involve methods for preparing a sequencing library enriched for duplexed DNA molecules. In some embodiments, the methods involve the use of a loop-shaped (hairpin) adapter and a rolling circle amplification (RCA) reaction to selectively amplify and enrich both the forward (+) and reverse (−) complement strands of double-stranded DNA molecules, thereby increasing duplex reads in sequencing. In the RCA reaction, both strands (positive (+) and negative (−) strands) of undamaged/repaired adapter-duplex DNA constructs are amplified. Adapter-duplex DNA constructs with unrepaired damage (e.g., nicked DNA) or incomplete adapter ligation are not amplified and no RCA product (duplex concatemer) is produced. In other embodiments, the methods involve the use of a loop-shaped (hairpin) adapter and subsequent nuclease digest step to remove (or deplete) undesired DNA molecules (e.g., nicked dsDNA and/or unligated DNA molecules). In still other embodiments, the methods utilize loop-shaped (hairpin) adapter ligation, a nuclease digestion, and a rolling circle amplification (RCA) reaction to selectively amplify and enrich double-stranded (or duplex) DNA molecules for subsequent sequencing.

In accordance with one embodiment of the present invention, a rolling circle amplification (RCA) reaction uses an original dsDNA molecule as template to produce multiple tandem (concatemeric) copies of both the positive (+) and negative (−) strands of the dsDNA molecule. Because the amplification reaction uses the original dsDNA molecules as a template to produce multiple copies, the recovery of duplex sequencing data (i.e., both the forward and reverse complement strands from a dsDNA molecule) is increased, allowing for improvements in subsequent error correction.

In some embodiments, the incubation time for an RCA reaction may be selected based on a desired target fragment size. For example, the incubation time for an RCA reaction may be selected to increase the efficiency of amplification of relatively small fragments (e.g., about 100 bp or less), thereby enriching for smaller target fragments.

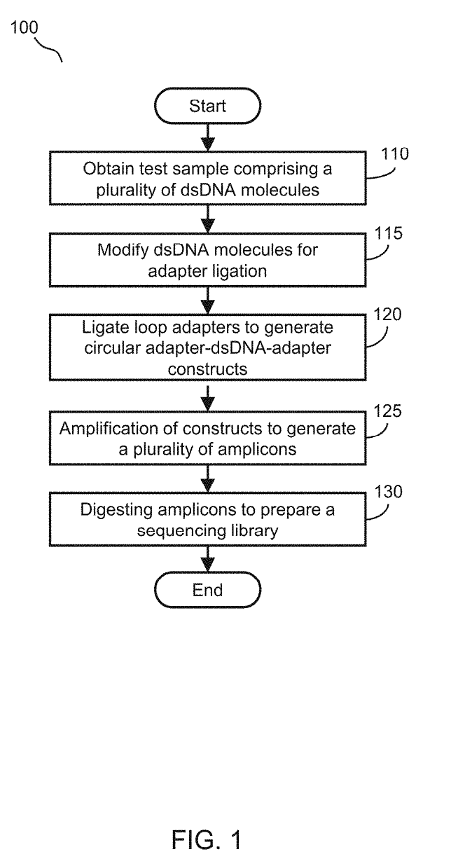

FIG. 1 is a flow diagram illustrating a method 100 of preparing a sequencing library enriched for duplexed DNA molecules, in accordance with one embodiment of the present invention. Method 100 uses a loop-shaped (or hairpin) adapter and a rolling circle amplification (RCA) reaction to selectively amplify a plurality of duplexed DNA fragments (i.e., both the forward (+) and reverse (−) complement strands), thereby enriching for duplexed DNA molecules in the sequencing library. Method 100 includes, but is not limited to, the following steps.

At step 110, a DNA test sample comprising a plurality of double-stranded DNA (dsDNA) molecules comprising a forward (+) strand sequence and a reverse (−) complement strand sequence are obtained from a subject (e.g., a patient). In one embodiment, the test sample may be a biological test sample selected from the group consisting of blood, plasma, serum, urine, saliva, fecal matter, and any combination thereof. In another embodiment, the test sample may be a biological test sample including one or more cells (e.g., blood cells). Alternatively, in still another embodiment, the test sample or biological test sample may comprise a test sample selected from the group consisting of whole blood, a blood fraction, a tissue biopsy, pleural fluid, pericardial fluid, cerebrospinal fluid (CSF), peritoneal fluid, and any combination thereof. In other embodiments, the sample is a plasma sample from a cancer patient, or a patient suspected of having cancer. In accordance with some embodiments, the test sample or biological test sample comprises a plurality of cell-free nucleic acids (e.g., cell-free DNA (cfDNA) and/or cell-free RNA (cfRNA)) fragments. In other embodiments, the test sample or biological test sample comprises a plurality of cell-free nucleic acid (e.g., cell-free DNA and RNA) fragments originating from healthy cells and from cancer cells. Optionally, in one embodiment, cell-free nucleic acids (e.g., cfDNA and/or cfRNA) can be extracted and/or purified from the test sample before proceeding with subsequent library preparation steps. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (Qiagen). In some embodiments, the sample can be, for example, a fragmented genomic DNA (gDNA) sample (e.g., a sheared gDNA sample).

At step 115, the double-stranded DNA (dsDNA) molecules are modified for adapter ligation. For example, the ends of dsDNA molecules are repaired using, for example, T4 DNA polymerase and/or Klenow polymerase and phosphorylated with a polynucleotide kinase enzyme prior to ligation of the adapters. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

At step 120, loop-shaped (hairpin) adapters are ligated to both ends of the dsDNA molecules to generate circular adapter-dsDNA-adapter molecule constructs. The ligation reaction can be performed using any suitable ligation step (e.g., using a ligase) which joins the dsDNA adapters to the dsDNA molecules to form circular adapter-dsDNA-adapter constructs. In one example, the ligation reaction is performed using T4 DNA ligase. In another example, T7 DNA ligase is used for adapter ligation to the dsDNA molecules.

The loop-shaped (hairpin) adapters may include, for example, a double-stranded stem region and a loop region comprising a primer binding site(s) and/or a recognition site for nuclease digestion (e.g., an endonuclease restriction site). In accordance with one aspect of the present invention, as described in more detail below, a primer binding site can be used to initiate subsequent rolling circle amplification (RCA) of the adapter-dsDNA-adapter constructs. A recognition site can be used to digest the single-stranded concatemeric RCA product (or concatemeric amplicon) into a plurality of forward (+) and reverse (−) complement strand sequences.

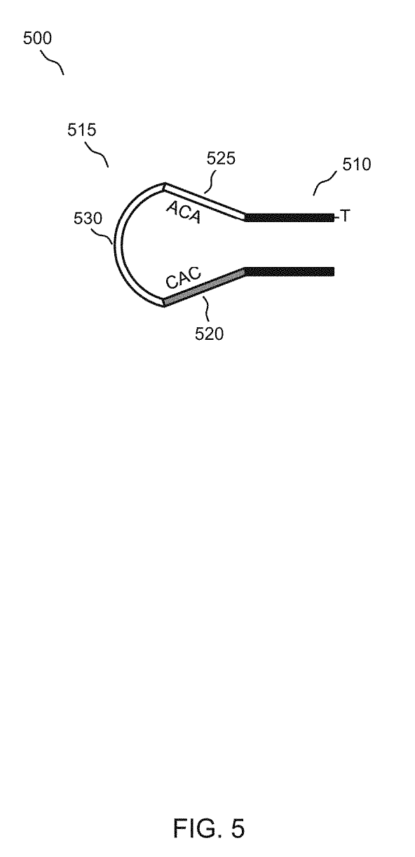

In one embodiment, the loop-shaped (hairpins) adapters can include a unique molecular identifier (UMI) sequence, such that, after library preparation, the sequencing library will include UMI-tagged amplicons derived from unique dsDNA molecules or dsDNA fragments. In one embodiment, unique sequence tags (e.g., unique molecular identifiers (UMIs)) can be used to identify unique nucleic acid sequences from a test sample. For example, differing unique sequence tags (UMIs) can be used to differentiate various unique nucleic acid sequence fragments originating from the test sample. In another embodiment, the UMI sequences can be used to identify duplex sequence reads from a dsDNA molecule (i.e., the single-strand forward (+) and single-strand reverse (−) complement strand sequences originating for a single dsDNA molecule). In still another embodiment, unique sequence tags (UMIs) can be used to reduce amplification bias, which is the asymmetric amplification of different targets due to differences in nucleic acid composition (e.g., high GC content). The unique sequence tags (UMIs) can also be used to discriminate between nucleic acid mutations that arise during amplification. In one embodiment, the unique sequence tag can comprise a short oligonucleotide sequence having a length of from about 2 nt to about 100 nt, from about 2 nt to about 60 nt, from about 2 to about 40 nt, or from about 2 to about 20 nt. In another embodiment, the UMI tag can comprise a short oligonucleotide sequence greater than about 5, 6, 7, 8, 9, 10, 11, 12, 13, 14, 15, 16, 17, or 18 nucleotides (nt) in length. An example of a loop-shaped or hairpin adapter useful in the practice of the present invention is described in more detail with reference to FIG. 5.

The unique sequence tags can be present in a multi-functional loop-shaped sequencing adapter. For example, the loop-shaped sequencing adapter can comprise a unique sequence tag, a sample-specific index sequence (or tag), and/or a universal priming site. In one embodiment, the sequencing adapters utilized may also include one or more sequencing oligonucleotides for use in subsequent cluster generation and/or sequencing (e.g., known P5 and P7 sequences for used in sequencing by synthesis (SBS) (IIlumina, San Diego, Calif.)). In another embodiment, the loop-shaped adapter includes a sample-specific index sequence, such that, after library preparation, the library can be combined with one or more other libraries prepared from individual samples, thereby allowing for multiplex sequencing. The sample-specific index sequence can comprise a short oligonucleotide sequence having a length of from about 2 nt to about 20 nt, from about 2 nt to about 10 nt, from about 2 to about 8 nt, or from about 2 to about 6 nt. In another embodiment, the sample-specific index sequence can comprise a short oligonucleotide sequence greater than about 2, 3, 4, 5, 6, 7, or 8 nucleotides (nt) in length.

At step 125, the circular adapter-dsDNA-adapter constructs generated in step 120 are amplified. For example, the circular adapter-dsDNA-adapter constructs can be amplified using a rolling circle amplification (RCA) reaction to generate a plurality of concatemer amplicons. In some embodiments, the plurality of concatemer amplicons (or concatemer RCA products) comprise alternating forward (+) and reverse (−) complement strand sequences originating from the dsDNA molecules. Amplification of the circular adapter-dsDNA-adapter constructs generated in step 120 enriches for undamaged (or repaired) fully ligated adapter-duplex DNA constructs. For example, undamaged (or repaired) duplexed molecules with adapters fully ligated at both ends can be amplified through RCA to generate a single-stranded tandem repeat (concatemer) comprising the forward (or positive) and reverse (or negative complementary) strand sequences. However, adapter-duplex DNA constructs with unrepaired damage (e.g., nicked DNA) or incomplete adapter ligation are not amplified, and thus, no RCA product is produced.

At step 130, a digestion step is used to digest the concatemer amplicons (i.e., the RCA product) into individual positive (+) and negative (−) complement strand sequences to generate a sequencing library. For example, as shown in FIG. 1, a sequence-specific restriction digestion reaction is performed to digest the concatemer amplicons (i.e., the RCA product) into individual positive (+) and negative (−) complement strand sequences generating a sequencing library. In general, any sequence-specific restriction method known in the art can be used to digest the concatemeric RCA product. For example, as shown in FIG. 5, a recognition site for nuclease digestion (e.g., an endonuclease restriction site) can be incorporated into the loop-shaped adapters and subsequently digested using a nuclease. In other embodiments, digestion of the concatemer amplicons (i.e., the RCA product) can be carried out using other methods known in the art. For example, the concatemer amplicons (i.e., the RCA product) can be used using a CRISPR-based method, a zinc-finger nuclease, or a transcription activator-like effector nuclease (TALEN).

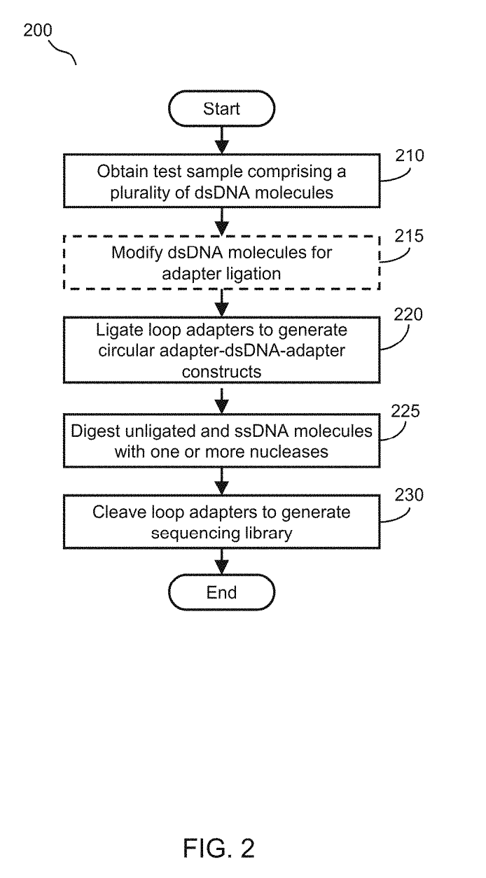

FIG. 2 is a flow diagram illustrating a method 200 of preparing a sequencing library enriched for duplexed DNA molecules, in accordance with another embodiment of the present invention. Method 200 uses a loop-shaped (or hairpin) adapter and a subsequent nuclease digestion step to enrich for a plurality of duplexed DNA fragments (i.e., both the forward (+) and reverse (−) complement strands), thereby enriching for duplexed DNA molecules in the sequencing library. Method 200 includes, but is not limited to, the following steps.

At step 210, a DNA test sample comprising a plurality of double-strand DNA (dsDNA) molecules comprising a forward (+) strand sequence and a reverse (−) complement strand sequence are obtained from a subject (e.g., a patient). As discussed in more detail elsewhere herein, the biological sample can be a blood, plasma, serum, urine, saliva samples, or any combination thereof. In another embodiment, the test sample may be a biological test sample including one or more cells (e.g., blood cells). Alternatively, in still another embodiment, the test sample or biological test sample may comprise a test sample selected from the group consisting of whole blood, a blood fraction, a tissue biopsy, pleural fluid, pericardial fluid, cerebrospinal fluid (CSF), peritoneal fluid, and any combination thereof. In other embodiments, the sample is a plasma sample from a cancer patient, or a patient suspected of having cancer. In accordance with some embodiments, the test sample or biological test sample comprises a plurality of cell-free nucleic acid (e.g., cell-free DNA (cfDNA) and/or cell-free RNA (cfRNA)) fragments. In other embodiments, the test sample or biological test sample comprises a plurality of cell-free nucleic acid (e.g., cell-free DNA and RNA) fragments originating from healthy cells and from cancer cells. Optionally, in one embodiment, cell-free nucleic acids (e.g., cfDNA and/or cfRNA) can be extracted and/or purified from the test sample before proceeding with subsequent library preparation steps. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (Qiagen). In some embodiments, the sample can be, for example, a fragmented genomic DNA (gDNA) sample (e.g., a sheared gDNA sample).

At step 215, optionally the double-stranded DNA (dsDNA) molecules are modified for adapter ligation. For example, the ends of dsDNA molecules can be repaired using, for example, T4 DNA polymerase and/or Klenow polymerase and phosphorylated with a polynucleotide kinase enzyme prior to ligation of the adapters. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

At step 220, loop-shaped (hairpin) adapters are ligated to both ends of the dsDNA molecules to generate circular adapter-dsDNA-adapter molecule constructs. The loop-shaped (hairpin) adapters may include, for example, a double-stranded stem region and a loop region comprising a primer binding sites and/or a recognition site for nuclease digestion (e.g., an endonuclease restriction site). As described elsewhere herein, the loop-shaped adapters (hairpin) can comprise a unique molecular identifier (UMI) and a sequence-specific recognition site (e.g., an endonuclease restriction site). Furthermore, as noted elsewhere in this disclosure, the loop-shaped adapters can also include one or more primer binding sites (e.g., universal primer sites) and/or one or more sequencing oligonucleotides for use in subsequent cluster generation and/or sequencing (e.g., known P5 and P7 sequences for used in sequencing by synthesis (SBS) (Illumina, San Diego, Calif.)). The ligation reaction can be performed using any suitable ligation step (e.g., using a ligase) which joins the dsDNA adapters to the dsDNA molecules to form circular adapter-dsDNA-adapter constructs. In one example, the ligation reaction is performed using T4 DNA ligase. In another example, T7 DNA ligase is used for adapter ligation to the dsDNA molecules.

As is well known in the art, current protocols for preparing a sequencing library from double-stranded DNA typically includes DNA end repair, 3′ end A-tailing, ligation of sequencing adapters to the double-stranded (duplexed) DNA, and polymerase chain reaction (PCR) amplification to enrich for adapter ligated DNA molecules. The procedure requires four successful ligation events to obtain sequenceable fragments for both the forward and reverse strands of a double-stranded DNA molecule. If a single ligation event fails during library preparation, one strand of the duplexed library fragment will not be amplified and a non-duplexed read will be observed during sequence analysis. As such, in accordance with this embodiment, at step 225, the sample comprising a plurality circular adapter-dsDNA-adapter constructs from step 220 is treated with one or more nucleases to remove (or deplete) undesired DNA molecules. For example, after ligation step 220, undesired single-stranded DNA (ssDNA) molecules and/or unligated double-stranded DNA (dsDNA) molecules can be digested, at step 225, using one or more ssDNA specific nucleases and/or dsDNA specific nucleases. In general, any known ssDNA or dsDNA nucleases can be used in the practice of the present invention. In one embodiment, the one or more ssDNA and/or dsDNA nucleases are 5′→3′ or 3′→5′ exonuclease that digest the DNA molecules from unligated ends. In another embodiment, the exonuclease is exonuclease V (RecBD) (New England BioLabs, Inc., Ipswich, Mass.). In still another embodiment, the exonuclease is T5 exonuclease (New England BioLabs, Inc., Ipswich, Mass.).

At step 230, the plurality of loop-shaped adapters are cleaved at the recognition site with a nuclease to generate a sequencing library comprising a plurality of double-stranded DNA molecules having adapters ligated to both ends. For example, a sequence-specific restriction digestion reaction is performed to digest the circular adapter-dsDNA-adapter molecule constructs into adapter-dsDNA-adapter constructs, thereby generating a sequencing library. As shown in FIG. 5, a recognition site for nuclease digestion (e.g., an endonuclease restriction site) can be incorporated into the loop-shaped adapters and digested using a nuclease. In general, any site-specific nuclease known in the art can be used to cleave the plurality of loop-shaped adapters at the recognition site. In one embodiment, the nuclease is an endonuclease that specifically cleaves at a known recognition site. In another embodiment, the recognition site is a uracil residue and the endonuclease is uracil-DNA glycosylase (UDG) (New England BioLabs, Inc., Ipswich, Mass.).

Optionally, the adapter-dsDNA-adapter constructs can be amplified to generate the sequencing library. For example, the adapter-dsDNA-adapter constructs can be amplified by PCR using a DNA polymerase and a reaction mixture containing one or more primers and/or a mixture of deoxyribonucleotide triphosphates (i.e., dNTPs).

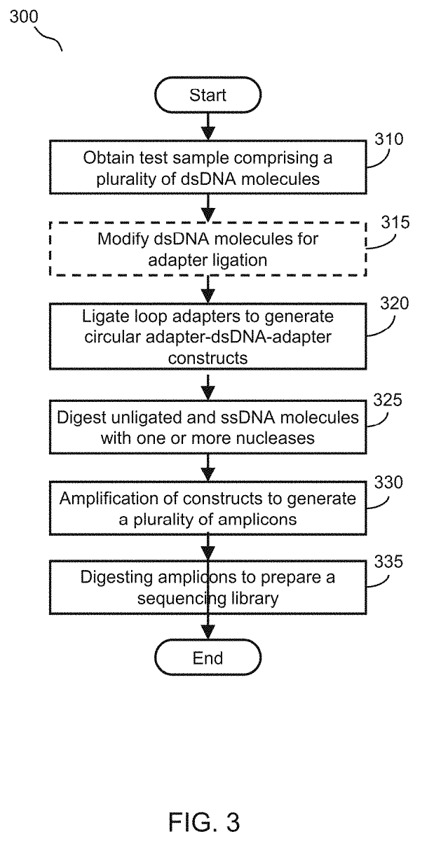

FIG. 3 is a flow diagram illustrating a method 300 of preparing a sequencing library enriched for duplexed DNA molecules, in accordance with another embodiment of the present invention. Method 300 uses a loop-shaped (or hairpin) adapter, a subsequent nuclease digestion step and an amplification step (e.g., rolling circle amplification (RCA)) to enrich for a plurality of duplexed DNA fragments (i.e., both the forward (+) and reverse (−) complement strands), thereby enriching for duplexed DNA molecules in the sequencing library. Method 300 includes, but is not limited to, the following steps.

At step 310, a DNA test sample comprising a plurality of double-stranded DNA (dsDNA) molecules comprising a forward (+) strand sequence and a reverse (−) complement strand sequence are obtained from a subject (e.g., a patient). As discussed in more detail elsewhere herein, the biological sample can be a blood, plasma, serum, urine, saliva samples, or any combination thereof. In another embodiment, the test sample may be a biological test sample including one or more cells (e.g., blood cells). Alternatively, in still another embodiment, the test sample or biological test sample may comprise a test sample selected from the group consisting of whole blood, a blood fraction, a tissue biopsy, pleural fluid, pericardial fluid, cerebrospinal fluid (CSF), peritoneal fluid, and any combination thereof. In other embodiments, the sample is a plasma sample from a cancer patient, or a patient suspected of having cancer. In accordance with some embodiments, the test sample or biological test sample comprises a plurality of cell-free nucleic acids (e.g., cell-free DNA (cfDNA) and/or cell-free RNA (cfRNA)) fragments. In other embodiments, the test sample or biological test sample comprises a plurality of cell-free nucleic acids (e.g., cell-free DNA and RNA) fragments originating from healthy cells and from cancer cells. Optionally, in one embodiment, cell-free nucleic acids (e.g., cfDNA and/or cfRNA) can be extracted and/or purified from the test sample before proceeding with subsequent library preparation steps. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (Qiagen). In some embodiments, the sample can be, for example, a fragmented genomic DNA (gDNA) sample (e.g., a sheared gDNA sample).

At step 315, optionally the double-stranded DNA (dsDNA) molecules are modified for adapter ligation. For example, the ends of dsDNA molecules can be repaired using, for example, T4 DNA polymerase and/or Klenow polymerase and phosphorylated with a polynucleotide kinase enzyme prior to ligation of the adapters. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

At step 320, loop-shaped (hairpin) adapters are ligated to both ends of the dsDNA molecules to generate circular adapter-dsDNA-adapter molecule constructs. The loop-shaped (hairpin) adapters may include, for example, a double-stranded stem region and a loop region comprising a primer binding site and/or a recognition site for nuclease digestion (e.g., an endonuclease restriction site). As described elsewhere herein, the loop-shaped adapters (hairpin) can comprise a unique molecular identifier (UMI) and a restriction site (e.g., an endonuclease restriction site). Furthermore, as noted elsewhere in this disclosure, the loop-shaped adapters can also include one or more primer binding sites (e.g., universal primer sites) and/or one or more sequencing oligonucleotides for use in subsequent cluster generation and/or sequencing (e.g., known P5 and P7 sequences for used in sequencing by synthesis (SBS) (Illumina, San Diego, Calif.)). In accordance with one aspect of the present invention, as described in more detail below, the primer binding site can be used to initiate subsequent rolling circle amplification (RCA) of the circular adapter-dsDNA-adapter constructs. The sequence-specific restriction site can be used to digest the single-stranded concatemeric RCA product (or concatemeric amplicon) into a plurality of forward (+) and reverse (−) complement strand sequences. The ligation reaction can be performed using any suitable ligation step (e.g., using a ligase) which joins the dsDNA adapters to the dsDNA molecules to form circular adapter-dsDNA-adapter constructs. In one example, the ligation reaction is performed using T4 DNA ligase. In another example, T7 DNA ligase is used for adapter ligation to the dsDNA molecules.

As noted above, current protocols for preparing a sequencing library from double-stranded DNA typically include DNA end repair, 3′ end A-tailing, ligation of sequencing adapters to the double-stranded (duplexed) DNA, and polymerase chain reaction (PCR) amplification to enrich for adapter ligated DNA molecules. The procedure requires four successful ligation events to obtain sequenceable fragments for both the forward and reverse strands of a double-stranded DNA molecule. If a single ligation event fails during library preparation, one strand of the duplexed library fragment will not be amplified and a non-duplexed read will be observed during sequence analysis. As such, in accordance with this embodiment, at step 325, the sample comprising a plurality circular adapter-dsDNA-adapter constructs from step 320 is treated with one or more nucleases to remove (or deplete) undesired DNA molecules. For example, after ligation step 320, undesired single-stranded DNA (ssDNA) molecules and/or unligated doubles-stranded DNA (dsDNA) molecules can be digested using ssDNA specific and/or dsDNA specific nuclease. In general, any known ssDNA or dsDNA nucleases can be used in the practice of the present invention. In one embodiment, the ssDNA and/or dsDNA nucleases are 5′→3′ or 3′→5′ exonuclease that digest the DNA molecules from unligated ends. In another embodiment, the exonuclease is exonuclease V (RecBD) (New England BioLabs, Inc., Ipswich, Mass.). In still another embodiment, the exonuclease is T5 exonuclease (New England BioLabs, Inc., Ipswich, Mass.).

In some embodiments, the incorporation of an exonuclease into a library preparation protocol results in a reduction in the number of collapsed reads, as described further in Example 1. Specifically, in certain embodiments, the incorporation of an exonuclease (e.g., a T5 exonuclease or an exonuclease V) decreases the number of collapsed reads by an amount that ranges from about 10% to about 80%, such as about 15%, 20%, 25%, 30%, 35%, 40%, 45%, 50%, 55%, 60%, 65%, 70% or about 75%. Aspects of the invention include methods that incorporate a pretreatment step or a repair step in which input DNA is pretreated and/or repaired before a library preparation procedure is conducted in order to rescue a reduction in collapsed reads.

In some embodiments, the incorporation of an exonuclease into a library preparation protocol results in an increase in the percentage of duplex DNA, as described further in Example 1. Specifically, in certain embodiments, the incorporation of an exonuclease (e.g., a T5 exonuclease or an exonuclease V) increases the percentage of duplex DNA by an amount that ranges from about 35% to about 75%, such as about 40%, 45%, 50%, 55%, 60%, 65% or 70%. In some embodiments, the incorporation of an exonuclease enzyme increases the percentage of duplex DNA yield from about 40% to about 75%, from about 45% to about 75%, or from about 45% to about 65% or to about 70%.

After the nuclease treatment step 325, at step 330, the circular adapter-dsDNA-adapter constructs generated in step 320 are amplified. For example, the circular adapter-dsDNA-adapter constructs can be amplified using a rolling circle amplification (RCA) reaction to generate a plurality of concatemer amplicons. In accordance with the present invention, the plurality of concatemer amplicons (or concatemer RCA products) comprise alternating forward (+) and reverse (−) complement strand sequences originating from the dsDNA molecules. Amplification of the circular adapter-dsDNA-adapter constructs generated in step 320 enriches for undamaged (or repaired) fully ligated adapter-duplex DNA constructs. For example, undamaged (or repaired) duplexed molecules with adapters fully ligated at both ends can be amplified through RCA to generate a single-stranded tandem repeat (concatemer) comprising the forward (or positive) and negative (or reverse complementary) strand sequences. However, adapter-duplex DNA constructs with unrepaired damage (e.g., nicked DNA) or incomplete adapter ligation are not amplified, and thus, no RCA product is produced.

At step 335, a restriction digestion reaction is performed to digest the concatemer amplicons (i.e., the RCA product) into individual positive (+) and negative (−) complement strand sequences generating a sequencing library. In general, any sequence-specific restriction method known in the art can be used to digest the concatemeric RCA product. For example, as shown in FIG. 3, a recognition site nuclease digestion (e.g., an endonuclease restriction site) can be incorporated into the loop-shaped adapters and subsequently digested using a nuclease.

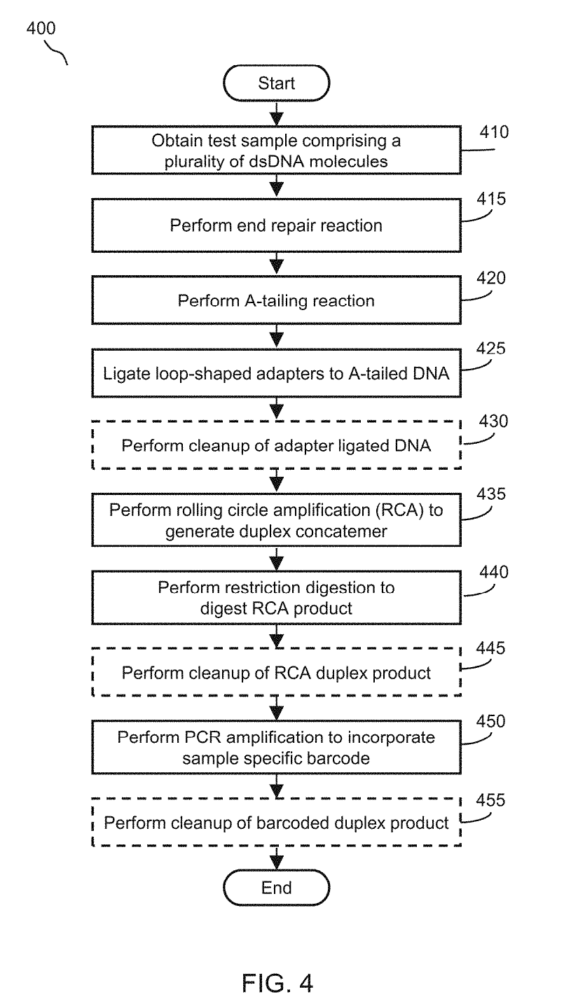

FIG. 4 is a flow diagram illustrating a method 400 of preparing a sequencing library enriched for duplexed DNA molecules, in accordance with another embodiment of the present invention. Method 400 uses a loop-shaped (or hairpin) adapter and a rolling circle amplification (RCA) reaction to selectively amplify a plurality of duplexed DNA fragments (i.e., both the forward (+) and reverse (−) complement strands), thereby enriching for duplexed DNA molecules in the sequencing library. Method 400 includes, but is not limited to, the following steps.

At step 410, a test sample comprising double-stranded DNA (dsDNA) molecules is obtained from a subject (e.g., a patient). As noted above, the test sample may be a biological test sample selected from the group consisting of blood, plasma, serum, urine, saliva, fecal matter, and any combination thereof. Alternatively, the test sample or biological test sample may comprise a test sample selected from the group consisting of whole blood, a blood fraction, a tissue biopsy, pleural fluid, pericardial fluid, cerebrospinal fluid (CSF), peritoneal fluid, and any combination thereof. In other embodiments, the sample is a plasma sample from a cancer patient, or a patient suspected of having cancer. In accordance with some embodiments, the test sample or biological test sample comprises a plurality of cell-free nucleic acid (e.g., cell-free DNA (cfDNA) and/or cell-free RNA (cfRNA)) fragments. In other embodiments, the test sample or biological test sample comprises a plurality of cell-free nucleic acid (e.g., cell-free DNA and RNA) fragments originating from healthy cells and from cancer cells. Optionally, in one embodiment, cell-free nucleic acids (e.g., cfDNA and/or cfRNA) can be extracted and/or purified from the test sample before proceeding with subsequent library preparation steps. The sample can be, for example, a cell-free DNA (cfDNA) sample or a fragmented genomic DNA (gDNA) sample (e.g., a sheared gDNA sample).

At step 415, the double-stranded DNA (dsDNA) molecules are modified for adapter ligation. For example, as shown in FIG. 4, the ends of dsDNA molecules are repaired using, for example, T4 DNA polymerase and/or Klenow polymerase and phosphorylated with a polynucleotide kinase enzyme prior to ligation of the adapters.

At step 420, a single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

At step 425, loop-shaped (hairpin) adapters are ligated to both ends of the dsDNA molecules to generate circular adapter-dsDNA-adapter molecule constructs. The ligation reaction can be performed using any suitable ligation step (e.g., using a ligase) which joins the dsDNA adapters to the dsDNA molecules to form circular adapter-dsDNA-adapter constructs. In one example, the ligation reaction is performed using T4 DNA ligase. In another example, T7 DNA ligase is used for adapter ligation to the dsDNA molecules. An example of a loop-shaped adapter useful in the practice of the present invention is described in more detail with reference to FIG. 5.

As described above, the loop-shaped adapters may include, for example, a double-stranded stem region and a loop region comprising a primer binding sites and/or a recognition site for nuclease digestion (e.g., an endonuclease restriction site). Furthermore, the loop-shaped (hairpins) adapters can include a unique molecular identifier (UMI) sequence, such that, after library preparation, the sequencing library will include UMI tagged amplicons derived from unique dsDNA molecules or dsDNA fragments, as described above in reference to FIG. 1.

At step 430, optionally, a cleanup protocol (e.g., an SPRI purification protocol) is performed to purify or isolate the circular adapter-dsDNA-adapter constructs from the reaction sample.

At step 435, the circular adapter-dsDNA-adapter constructs generated in step 425 are amplified. For example, the circular adapter-dsDNA-adapter constructs can be amplified using a rolling circle amplification (RCA) reaction to generate a plurality of concatemer amplicons. In accordance with the present invention, the plurality of concatemer amplicons (or concatemer RCA products) comprise alternating forward (+) and reverse (−) complement strand sequences originating from the dsDNA molecules. Amplification of the circular adapter-dsDNA-adapter constructs generated in step 425 enriches for undamaged (or repaired) fully ligated adapter-duplex DNA constructs. For example, undamaged (or repaired) duplexed molecules with adapters fully ligated at both end can be amplified through RCA to generate a single-stranded tandem repeat (concatemer) comprising the forward (or positive) and reverse (or negative complementary) strand sequences. However, adapter-duplex DNA constructs with unrepaired damage (e.g., nicked DNA) or incomplete adapter ligation are not amplified, and thus, no RCA product is produced.

At step 440, a restriction digestion reaction is performed to digest the concatemer amplicons (i.e., the RCA product) into individual, single-stranded positive (+) and negative (−) complement strand sequences. In general, any sequence-specific restriction method known in the art can be used to digest the concatemeric RCA product. For example, as shown in FIG. 5, a recognition site for nuclease digestion (e.g., an endonuclease restriction site) can be incorporated into the loop-shaped adapters and subsequently digested using a nuclease.

At step 445, optionally, a cleanup protocol (e.g., an SPRI purification protocol) is performed to purify or isolate the single-stranded positive (+) and negative (−) complement strand sequences from the reaction sample.

At step 450, a sample-specific indexing (barcoding) sequence is added, providing each sample a sample-specific sample index (barcode) sequence allowing for multiplexing. For example, as is well known in the art, one or more primers can be used in a PCR amplification step to add a sample-specific indexing or barcode sequence to the single-stranded positive (+) and negative (−) complement strand sequences obtained from step 440.

At step 455, optionally, a cleanup protocol (e.g., an SPRI purification protocol) is performed to purify or isolate the indexed single-stranded positive (+) and negative (−) complement strand sequences to generate a sequencing library.

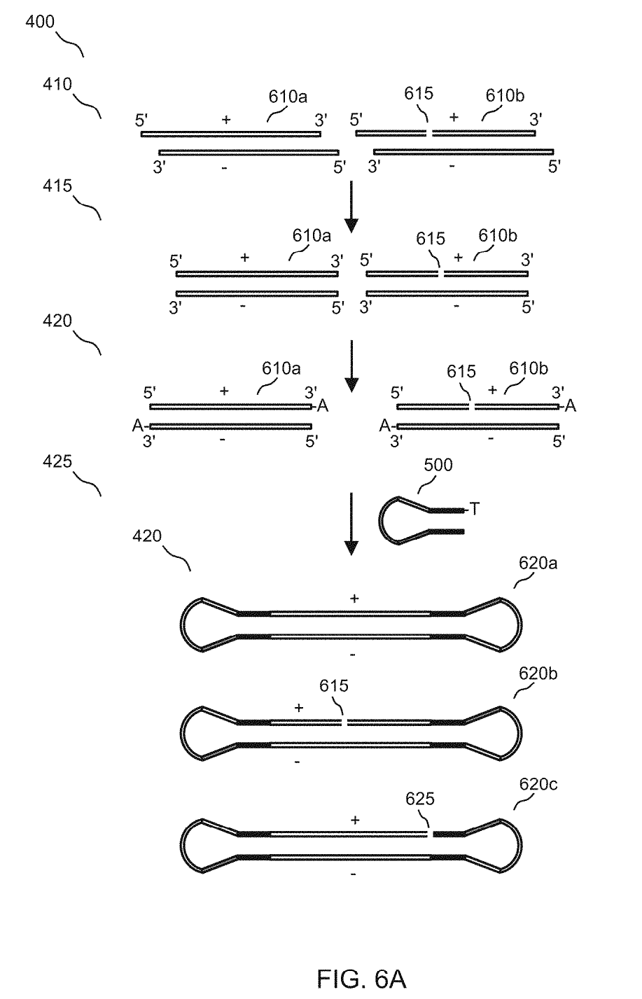

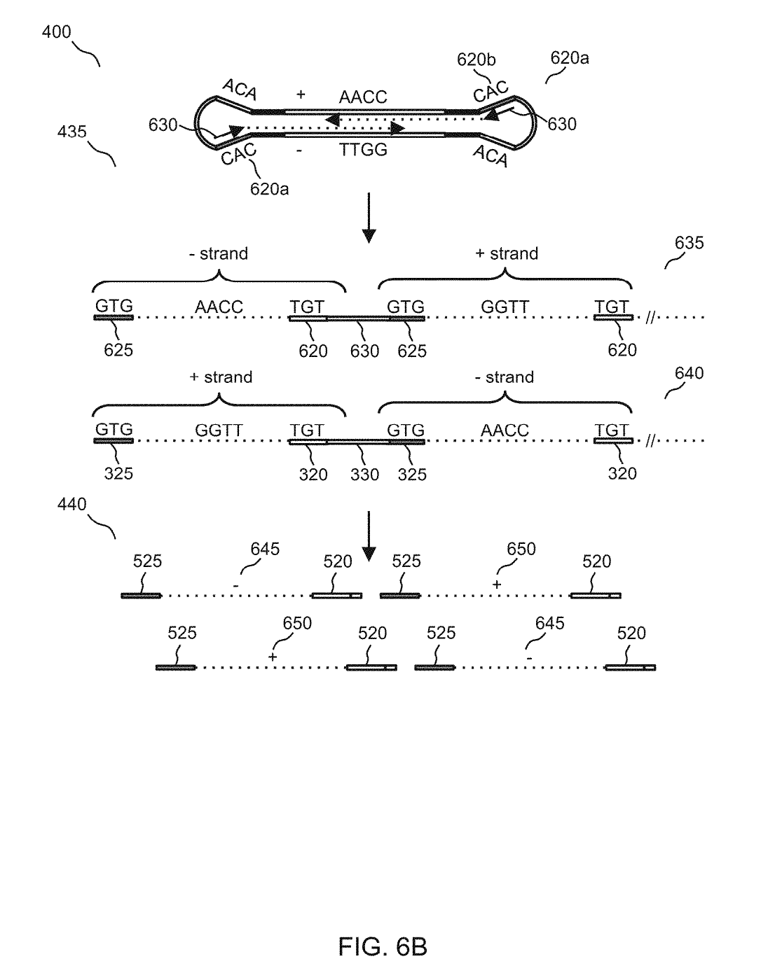

FIG. 5 is a schematic diagram illustrating a loop-shaped (or hairpin) adapter 500 useful in the practice of the present invention for amplification and enrichment of a plurality of duplexed DNA fragments (i.e., both the forward (+) and reverse (−) complement strands) in sequencing library preparation. Loop-shaped adapter 500 includes a double stranded stem region 510 and a loop region 515. In one embodiment, as described elsewhere, stem region 510 can include a single nucleotide (e.g., as shown a “T” nucleotide), that is complementary to a single base 3′ overhang on the dsDNA molecule. The stem region can also include a unique molecular identifier (UMI), as described elsewhere in the present application. Loop region 515 can include a first region 520, a second region 525, and a nuclease recognition site 530. First region 520 and second region 525 can include primer binding sites to initiate rolling circle amplification (RCA) on ligated adapter-dsDNA-adapter constructs. Nuclease recognition site 530 can include a sequence-specific restriction site allowing for digestion of the concatemer amplicons (i.e., the RCA product) into individual, single-stranded positive (+) and negative (−) complement strand sequences. Digestion of a linear single-stranded concatemer RCA product is further described with reference to FIG. 6.

FIGS. 6A and 6B provide schematic diagrams showing pictorially some of the steps of method 400 of FIG. 4. Namely, at step 410, a test sample comprising a plurality of double-stranded DNA (dsDNA) molecules is obtained from a test subject (e.g., a patient). Referring to FIG. 6A, two dsDNA molecules 610 (i.e., an undamaged DNA molecule 610a and a damaged DNA molecule 610b) with 5′ end overhangs are shown. Furthermore, as shown, dsDNA molecule 610b includes a nick (or gap) 615.

At step 415, an enzymatic repair reaction is performed to repair damage and convert the 5′ overhangs on DNA molecules 610a and 610b to blunt-ends for subsequent A-tailing and adapter ligation.

At step 420, an A-tailing reaction is performed to add a single “A” nucleotide to the 3′ ends of the blunt-ended DNA molecules 610a and 610b producing a one base 3′ overhang that is complementary to the one base 3′ T base overhang on double-stranded stem region 510 of stem-loop adapter 500 described with reference to FIG. 5.

At step 425, loop-shaped adapter 500 is ligated to the ends of the A-tailed dsDNA molecules 610a and 610b. The ligation reaction can be performed using any suitable ligation step (e.g., using T4 DNA ligase) which joins a copy of the loop-shaped adapter 500 to both ends of the dsDNA molecules 610a and 610b to form a circular adapter-dsDNA-adapter construct 620 (comprising loop-shaped adapter 500—dsDNA molecule 610—loop-shaped adapter 500). In this example, three adapter-dsDNA-adapter constructs 620 are shown: (1) a fully ligated undamaged adapter-dsDNA-adapter construct 620a (i.e., all strands (+ and −) are ligated to adapters); (2) a fully ligated nicked (damaged) adapter-dsDNA-adapter construct 620b; and (3) a partially ligated undamaged adapter-dsDNA-adapter construct 620c, wherein an incomplete ligation reaction (or adapter ligation failure) creates a gap 625 at a dsDNA/adapter junction.

At step 435, the circular adapter-dsDNA-adapter constructs are amplified using a rolling circle application (RCA) reaction to generate a plurality of single-stranded concatemer amplicons (or concatemer RCA products) comprise alternating forward (+) and reverse (−) complement strand sequences originating from the dsDNA molecules. For example, as shown, an RCA primer 630 that is complementary to primer binding sites in the first regions 520a and 520b is used to initiate rolling circle amplification (RCA), generating single-stranded concatemeric amplicons 635 and 640, respectively, comprising alternating forward (+ strand) and reverse (−strand) complement strand sequences separated by sequence-specific restriction site 530. Adapter-dsDNA-adapter construct 620b with unrepaired damage (i.e., nick 615) and adapter-dsDNA-adapter construct 620c with incomplete adapter ligation (i.e., gap 625) are not amplified, and thus, no concatemer (RCA product) is produced.

At step 440, a sequence-specific restriction digestion is performed to cleave or digest the concatemer amplicons 635 and 640 at nuclease recognition sites 530, thereby generating a plurality of individual single-stranded forward strand (+ strand) fragments 645 and reverse (−strand) complement strand fragments 650.

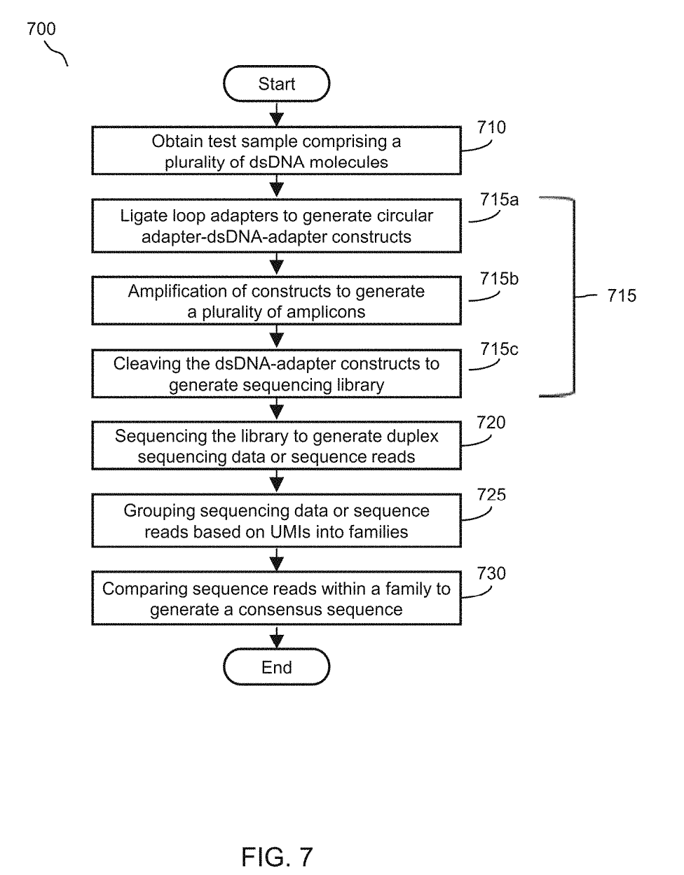

FIG. 7 is a flow diagram illustrating a method 700 for preparing an improved sequencing library for duplex sequencing based error correction.

As shown in FIG. 7, at step 710, a biological test sample comprising a plurality of double-stranded DNA (dsDNA) molecules is obtained from a subject (e.g., a patient known to have or suspected of having cancer). As discussed in more detail elsewhere herein, the biological sample can be a blood, plasma, serum, urine, saliva samples, or any combination thereof. Alternatively, as noted above, the biological sample can be a whole blood, a blood fraction, a tissue biopsy, a pleural fluid, pericardial fluid, a cerebrospinal fluid (CSF), a peritoneal fluid, or any combination thereof. In accordance with some embodiments, the biological test sample can comprise a plurality of cell-free nucleic acids (e.g., cell-free DNA (cfDNA)) fragments. In some embodiments, the sample is a plasma sample from a cancer patient, or a patient suspected of having cancer. Optionally, the cell-free nucleic acids (e.g., cfDNA) can be extracted and/or purified from the biological test sample using any means known in the art.

Optionally, the double-stranded DNA (dsDNA) molecules are modified for adapter ligation. For example, the ends of dsDNA molecules are repaired using, for example, T4 DNA polymerase and/or Klenow polymerase and phosphorylated with a polynucleotide kinase enzyme prior to ligation of the adapters. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

At step 715, a sequencing library is prepared. For example, in one embodiment, a sequencing library can be prepared by ligating a loop-shaped (or hairpin) adapter to double-stranded DNA molecules in a test sample followed by rolling circle amplification (RCA) reaction to selectively amplify a plurality of duplexed DNA fragments (i.e., both the forward (+) and reverse (−) complement strands), thereby enriching for duplexed DNA molecules in the sequencing library (as described above in conjunction with FIG. 1). In another embodiment, a sequencing library can be prepared by ligating a loop-shaped (or hairpin) adapter to double-stranded DNA molecules in a test sample followed by a subsequent nuclease digestion step to enrich for a plurality of duplexed DNA fragments (as described above in conjunction with FIG. 2). In still another embodiment, a sequencing library can be prepared by ligating a loop-shaped (or hairpin) adapter to double-stranded DNA molecules in a test sample followed by a subsequent nuclease digestion step and an amplification step (e.g., rolling circle amplification (RCA)) to selectively amplify a plurality of duplexed DNA fragments (i.e., both the forward (+) and reverse (−) complement strands), thereby enriching for duplexed DNA molecules in the sequencing library (as described above in conjunction with FIG. 3).

For example, as shown at step 715a loop-shaped (hairpin) adapters are ligated to both ends of the dsDNA molecules to generate circular adapter-dsDNA-adapter molecule constructs. The ligation reaction can be performed using any suitable ligation step (e.g., using a ligase) which joins the dsDNA adapters to the dsDNA molecules to form circular adapter-dsDNA-adapter constructs. In one example, the ligation reaction is performed using T4 DNA ligase. In another example, T7 DNA ligase is used for adapter ligation to the dsDNA molecules. As described elsewhere herein, the loop-shaped adapters (hairpin) can comprise a unique molecular identifier (UMI) and a recognition site for nuclease digestion (e.g., an endonuclease restriction site). Furthermore, as noted elsewhere in this disclosure, the loop-shaped adapters can also include one or more primer binding sites (e.g., universal primer sites) and/or one or more sequencing oligonucleotides for use in subsequent cluster generation and/or sequencing (e.g., known P5 and P7 sequences for used in sequencing by synthesis (SBS) (Illumina, San Diego, Calif.)).

At step 715b, the circular adapter-dsDNA-adapter constructs generated in step 715a are amplified. For example, the circular adapter-dsDNA-adapter constructs can be amplified using a rolling circle amplification (RCA) reaction to generate a plurality of concatemer amplicons. In accordance with the present invention, the plurality of concatemer amplicons (or concatemer RCA products) comprise alternating forward (+) and reverse (−) complement strand sequences originating from the dsDNA molecules. Amplification of the circular adapter-dsDNA-adapter constructs generated in step 715a enriches for undamaged (or repaired) fully ligated adapter-duplex DNA constructs. For example, undamaged (or repaired) duplexed molecules with adapters fully ligated at both ends can be amplified through RCA to generate a single-stranded tandem repeat (concatemer) comprising the forward (or positive) and negative (or reverse complementary) strand sequences. However, adapter-duplex DNA constructs with unrepaired damage (e.g., nicked DNA) or incomplete adapter ligation are not amplified, and thus, no RCA product is produced.

At step 715c, a sequence-specific restriction digestion reaction is performed to digest the concatemer amplicons (i.e., the RCA product) into individual positive (+) and negative (−) complement strand sequences generating a sequencing library. In general, any sequence-specific restriction method known in the art can be used to digest the concatemeric RCA product.

At step 720, at least a portion of the sequencing library prepared in step 715 is sequenced to obtain sequencing data or sequence reads. In general, any method known in the art can be used to obtain sequence data or sequence reads from the sequencing library. For example, in one embodiment, sequencing data or sequence reads from the sequencing library can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some embodiments, sequencing is massively parallel sequencing using sequencing-by-synthesis with reversible dye terminators. In other embodiments, sequencing is sequencing-by-ligation. In yet other embodiments, sequencing is single molecule sequencing. In still another embodiment, sequencing is paired-end sequencing. Optionally, an amplification step can be performed prior to sequencing. In some embodiments, digestion of the concatemer amplicons (the RCA product) generating a plurality of single-stranded DNA (ssDNA) molecules comprising the forward (+ strand) and reverse (−strand) complementary strands from the original dsDNA molecules. These dsDNA molecule constructs (i.e., containing both the forward strand and reverse complement strand) allow for sequencing of both the forward and reverse complement strands, thereby simplifying identification of the associated forward strand and reverse complement strand from an original dsDNA fragment from the test sample.

As shown in FIG. 7, at step 725, sequencing data or sequence reads are grouped into families based on their unique molecular identifiers (UMIs). As used herein, a “family group” comprises a plurality of sequence reads identified, based on their associated UMIs, as originating from a single double-stranded DNA (dsDNA) molecule from the test sample. A “family” of sequence reads, as used herein, includes both a set of sequence reads originating from a specific forward (+) strand sequence and a set of sequence reads originating from the reverse (−) complement strand sequence (i.e., the forward strand and reverse complement from a single dsDNA molecule). For example, a family of sequence reads can be placed into a family group, where each of the sequence reads has either the same UMI (e.g., on a set of forward strands), or the reverse complement of the UMI sequence (e.g., on a set of reverse complement strands).

At step 730, the sequence reads within a family are compared to generate a consensus sequence. For example, the nucleotide base sequence for each of the plurality of sequence reads in a family (originating from both the forward strands and reverse complement strands) can be compared to determine the most probable nucleotide base at each position along the sequence. As used herein, a “consensus sequence” comprises a sequence of nucleotide bases identified as the most probable at each position along the sequence. In one embodiment, the consensus sequence comprises a sequence of nucleotide bases, wherein each base is identified as the most probable nucleotide base at a given position when a specific base is present at the position in a majority of the sequence reads within a family (i.e., from a plurality of sequence reads derived from both the forward and reverse complement strands within a family). In other embodiments, the consensus sequence comprises a sequence of nucleotide bases, wherein each base is identified as the most probable nucleotide base at a given position when a specific base is present at the position in at least 60%, at least 70%, at least 80%, at least 90%, or at least 95%, of the family members. In accordance with one embodiment, errors introduced during sample preparation and sequencing can be identified, and eliminated through the generation of a consensus sequence.

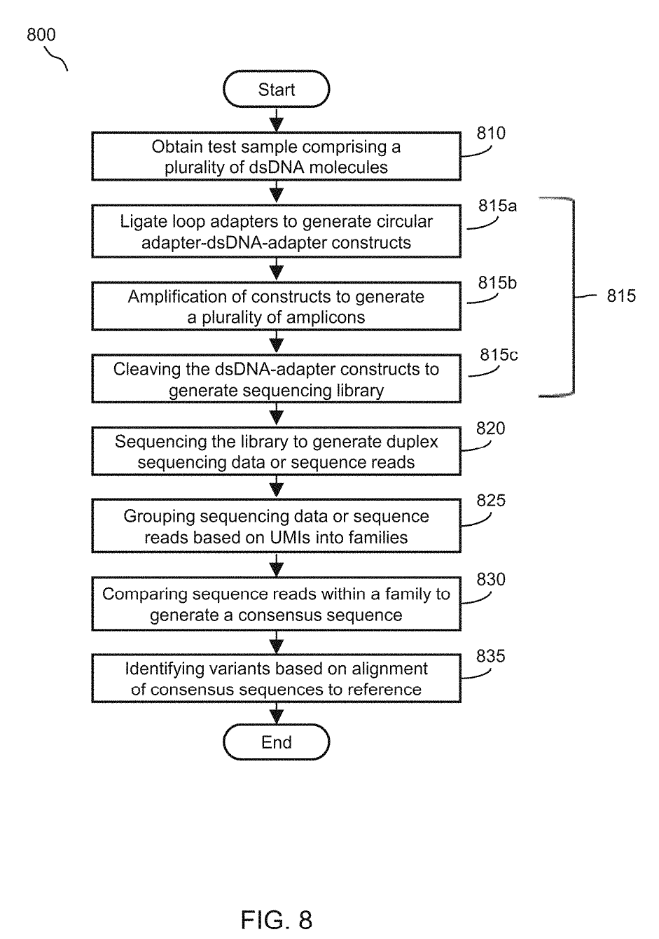

FIG. 8 is a flow diagram illustrating a method 800 for preparing an improved sequencing library for duplex sequencing based rare variant detection.

As shown in FIG. 8, at step 810, a biological test sample comprising a plurality of double-stranded DNA (dsDNA) molecules is obtained from a subject (e.g., a patient known to have or suspected of having cancer). As discussed in more detail elsewhere herein, the biological sample can be a blood, plasma, serum, urine, saliva samples, or any combination thereof. Alternatively, as noted above, the biological sample can be a whole blood, a blood fraction, a tissue biopsy, a pleural fluid, pericardial fluid, a cerebrospinal fluid (CSF), a peritoneal fluid, or any combination thereof. In accordance with some embodiments, the biological test sample can comprise a plurality of cell-free nucleic acid (e.g., cell-free DNA (cfDNA)) fragments. In some embodiments, the sample is a plasma sample from a cancer patient, or a patient suspected of having cancer. Optionally, the cell-free nucleic acids (e.g., cfDNA) can be extracted and/or purified from the biological test sample using any means known in the art.

Optionally, the double-stranded DNA (dsDNA) molecules are modified for adapter ligation. For example, the ends of dsDNA molecules are repaired using, for example, T4 DNA polymerase and/or Klenow polymerase and phosphorylated with a polynucleotide kinase enzyme prior to ligation of the adapters. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

At step 815, a sequencing library is prepared. For example, in one embodiment, a sequencing library can be prepared by ligating a loop-shaped (or hairpin) adapter to double-stranded DNA molecules in a test sample followed by rolling circle amplification (RCA) reaction to selectively amplify a plurality of duplexed DNA fragments (i.e., both the forward (+) and reverse (−) complement strands), thereby enriching for duplexed DNA molecules in the sequencing library (as described above in conjunction with FIG. 1). In another embodiment, a sequencing library can be prepared by ligating a loop-shaped (or hairpin) adapter to double-stranded DNA molecules in a test sample followed by a subsequent nuclease digestion step to enrich for a plurality of duplexed DNA fragments (as described above in conjunction with FIG. 2). In still another embodiment, a sequencing library can be prepared by ligating a loop-shaped (or hairpin) adapter to double-stranded DNA molecules in a test sample followed by a subsequent nuclease digestion step and an amplification step (e.g., rolling circle amplification (RCA)) to selectively amplify a plurality of duplexed DNA fragments (i.e., both the forward (+) and reverse (−) complement strands), thereby enriching for duplexed DNA molecules in the sequencing library (as described above in conjunction with FIG. 3).

At step 815a, loop-shaped (hairpin) adapters are ligated to both ends of the dsDNA molecules to generate circular adapter-dsDNA-adapter molecule constructs. The ligation reaction can be performed using any suitable ligation step (e.g., using a ligase) which joins the dsDNA adapters to the dsDNA molecules to form circular adapter-dsDNA-adapter constructs. In one example, the ligation reaction is performed using T4 DNA ligase. In another example, T7 DNA ligase is used for adapter ligation to the dsDNA molecules. As described elsewhere herein, the loop-shaped adapters (hairpin) can comprise a unique molecular identifier (UMI) and a recognition site for nuclease digestion (e.g., an endonuclease restriction site). Furthermore, as noted elsewhere in this disclosure, the loop-shaped adapters can also include one or more primer binding sites (e.g., universal primer sites) and/or one or more sequencing oligonucleotides for use in subsequent cluster generation and/or sequencing (e.g., known P5 and P7 sequences for used in sequencing by synthesis (SBS) (Illumina, San Diego, Calif.)).

At step 815b, the circular adapter-dsDNA-adapter constructs generated in step 815a are amplified. For example, the circular adapter-dsDNA-adapter constructs can be amplified using a rolling circle amplification (RCA) reaction to generate a plurality of concatemer amplicons. In some embodiments, the plurality of concatemer amplicons (or concatemer RCA products) comprise alternating forward (+) and reverse (−) complement strand sequences originating from the dsDNA molecules. Amplification of the circular adapter-dsDNA-adapter constructs generated in step 815 enriches for undamaged (or repaired) fully ligated adapter-duplex DNA constructs. For example, undamaged (or repaired) duplexed molecules with adapters fully ligated at both ends can be amplified through RCA to generate a single-stranded tandem repeat (concatemer) comprising the forward (or positive) and negative (or reverse complementary) strand sequences. However, adapter-duplex DNA constructs with unrepaired damage (e.g., nicked DNA) or incomplete adapter ligation are not amplified, and thus, no RCA product is produced.

At step 815c, a sequence-specific restriction digestion reaction is performed to digest the concatemer amplicons (i.e., the RCA product) into individual positive (+) and negative (−) complement strand sequences generating a sequencing library. In general, any sequence-specific restriction method known in the art can be used to digest the concatemeric RCA product.

At step 820, at least a portion of the sequence library prepared in step 815 is sequenced to obtain sequencing data or sequence reads. In general, any method known in the art can be used to obtain sequence data or sequence reads from the sequencing library. For example, in one embodiment, sequencing data or sequence reads from the sequencing library can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some embodiments, sequencing is massively parallel sequencing using sequencing-by-synthesis with reversible dye terminators. In other embodiments, sequencing is sequencing-by-ligation. In yet other embodiments, sequencing is single molecule sequencing. In still another embodiment, sequencing is paired-end sequencing. Optionally, an amplification step can be performed prior to sequencing. In some embodiments, digestion of the concatemer amplicons (the RCA product) generates a plurality of single-stranded DNA (ssDNA) molecules comprising the forward (+ strand) and reverse (−strand) complementary strands from the original dsDNA molecules. These dsDNA molecule constructs allow for sequencing of both the forward and reverse complement strands, thereby simplifying identification of the associated forward strand and reverse complement strand from an original dsDNA fragment from the test sample.

As shown in FIG. 8, at step 825, sequencing data or sequence reads are grouped into families based on their unique molecular identifiers (UMIs). As used herein, a “family group” comprises a plurality of sequence reads identified, based on their associated UMIs, as originating from a single double-stranded DNA (dsDNA) molecule from the test sample. A “family” of sequence reads, as used herein, includes both a set of sequence reads originating from a specific forward strand and a set of sequence reads originating from the reverse complement strand (i.e., the forward strand and reverse complement from a single dsDNA molecule). For example, a family of sequence reads can be placed into a family group, where each of the sequence reads has either the same UMI (e.g., on a set of forward strands), or the reverse complement of the UMI sequence (e.g., on a set of reverse complement strands).

At step 830, the sequence reads within a family are compared to generate a consensus sequence. For example, the nucleotide base sequence for each of the plurality of sequence reads in a family (originating from both the forward strands and reverse complement strands) can be compared to determine the most probable nucleotide base at each position along the sequence. As used herein, a “consensus sequence” comprises a sequence of nucleotide bases identified as the most probable at each position along the sequence. In one embodiment, the consensus sequence comprises a sequence of nucleotide bases, wherein each base is identified as the most probable nucleotide base at a given position when a specific base is present at the position in a majority of the sequence reads within a family (i.e., from a plurality of sequence reads derived from both the forward and reverse complement strands within a family). In other embodiments, the consensus sequence comprises a sequence of nucleotide bases, wherein each base is identified as the most probable nucleotide base at a given position when a specific base is present at the position in at least 60%, at least 70%, at least 80%, at least 90%, or at least 95%, of the family members. In accordance with one embodiment, errors introduced during sample preparation and sequencing can be identified, and eliminated through the generation of a consensus sequence.

Finally, as shown at step 835, the consensus sequence can be compared to, or aligned to, a reference sequence to identify a rare variant or mutation. For example, a rare variant or mutation can be identified where the consensus sequence varies at one or more nucleotide base positions compared to the reference sequence. Rare variants and/or mutations may include, for example, genetic alterations such as a somatic point mutation(s) (e.g., single nucleotide variations (SNVs)), somatic indels, and/or a somatic copy number alteration(s) (SCNA; e.g., amplification(s) and/or deletion(s)). In some embodiments, the somatic point mutation(s) (e.g., single nucleotide variations (SNVs)), somatic indels, and/or a somatic copy number alteration(s) (SCNA; e.g., amplification(s) and/or deletion(s)) may be tumor-derived. In accordance with one embodiment of the present invention, one or more rare variants and/or mutations identified herein can be used for detecting the presence or absence of cancer, determining cancer stage, monitoring cancer progression, and/or for determining a cancer classification (e.g., cancer type or cancer tissue of origin). In another embodiment, the sequencing data or sequence reads can be used to infer the presence or absence of cancer, cancer status and/or a cancer classification.

Aspects of the invention include methods that involve the incorporation of a topoisomerase enzyme into a library preparation protocol. Topoisomerases are known in the art, and can be used to reduce or eliminate supercoiling of DNA, which can be an impediment to RCA reactions. In certain embodiments, the incorporation of a topoisomerase enzyme increases RCA efficiency by an amount that ranges from about 10% to about 50%, such as about 15%, 20%, 25%, 30%, 35%, 40% or about 45%. In certain embodiments, the incorporation of a topoisomerase enzyme increases RCA efficiency by an amount that ranges from about 50% to about 500%, such as about 60%, 70%, 80%, 90%, 100%, 125%, 150%, 175%, 200%, 225%, 250%, 275%, 300%, 325%, 350%, 375%, 400%, 425%, 450%, or 475%.

In some embodiments, the concentration of a topoisomerase enzyme included in a reaction mixture ranges from about 0.1 U to about 2 U, such as about 0.2, 0.3, 0.4, 0.5, 0.6, 0.7, 0.8, 0.9, 1.0, 1.1, 1.2, 1.3, 1.4, 1.5, 1.6, 1.7, 1.8 or 1.9 U.

In one embodiment, one or more rare variants and/or mutations can be analyzed to detect the presence or absence of, determine the stage of, monitor progression of, and/or classify a carcinoma, a sarcoma, a myeloma, a leukemia, a lymphoma, a blastoma, a germ cell tumor, or any combination thereof. In some embodiments, the carcinoma may be an adenocarcinoma. In other embodiments, the carcinoma may be a squamous cell carcinoma. In still other embodiments, the carcinoma is selected from the group consisting of: small cell lung cancer, non-small-cell lung cancer (NSCLC), nasopharyngeal, colorectal, anal, liver, urinary bladder, cervical, testicular, ovarian, gastric, esophageal, head-and-neck, pancreatic, prostate, renal, thyroid, melanoma, and breast carcinoma. In another embodiment, one or more rare variants and/or mutations can be analyzed to detect a presence or absence of, determine the stage of, monitor progression of, and/or classify a sarcoma. In certain embodiments, the sarcoma can be selected from the group consisting of: osteosarcoma, chondrosarcoma, leiomyosarcoma, rhabdomyosarcoma, mesothelial sarcoma (mesothelioma), fibrosarcoma, angiosarcoma, liposarcoma, glioma, and astrocytoma. In still another embodiment, the one or more rare variants and/or mutations can be analyzed to detect the presence or absence of, determine the stage of, monitor progression of, and/or classify leukemia. In certain embodiments, the leukemia can be selected from the group consisting of: myelogenous, granulocytic, lymphatic, lymphocytic, and lymphoblastic leukemia. In still another embodiment, the one or more rare variants and/or mutations can be used to detect presence or absence of, determine the stage of, monitor progression of, and/or classify a lymphoma. In certain embodiments, the lymphoma can be selected from the group consisting of: Hodgkin's lymphoma and Non-Hodgkin's lymphoma.

### Sequencing and Bioinformatics

As reviewed above, aspects of the invention include sequencing of nucleic acid molecules to generate a plurality of sequence reads, compilation of a plurality of sequence reads into a sequencing library, and bioinformatic manipulation of the sequence reads and/or sequencing library to determine sequence information from a test sample (e.g., a biological sample). In some embodiments, one or more aspects of the subject methods are conducted using a suitably-programmed computer system, as described further herein.

In certain embodiments, a sample is collected from a subject, followed by enrichment for genetic regions or genetic fragments of interest. For example, in some embodiments, a sample can be enriched by hybridization to a nucleotide array comprising cancer-related genes or gene fragments of interest. In some embodiments, a sample can be enriched for genes of interest (e.g., cancer-associated genes) using other methods known in the art, such as hybrid capture. See, e.g., Lapidus (U.S. Pat. No. 7,666,593), the contents of which is incorporated by reference herein in its entirety. In one hybrid capture method, a solution-based hybridization method is used that includes the use of biotinylated oligonucleotides and streptavidin coated magnetic beads. See, e.g., Duncavage et al., J Mol Diagn. 13(3): 325-333 (2011); and Newman et al., Nat Med. 20(5): 548-554 (2014). Isolation of nucleic acid from a sample in accordance with the methods of the invention can be done according to any method known in the art.

Sequencing may be by any method or combination of methods known in the art. For example, known DNA sequencing techniques include, but are not limited to, classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, Polony sequencing, and SOLiD sequencing. Sequencing of separated molecules has more recently been demonstrated by sequential or single extension reactions using polymerases or ligases as well as by single or sequential differential hybridizations with libraries of probes.

One conventional method to perform sequencing is by chain termination and gel separation, as described by Sanger et al., Proc Natl. Acad. Sci. USA, 74(12): 5463 67 (1977), the contents of which are incorporated by reference herein in their entirety. Another conventional sequencing method involves chemical degradation of nucleic acid fragments. See, Maxam et al., Proc. Natl. Acad. Sci., 74: 560 564 (1977), the contents of which are incorporated by reference herein in their entirety. Methods have also been developed based upon sequencing by hybridization. See, e.g., Harris et al., (U.S. patent application number 2009/0156412), the contents of which are incorporated by reference herein in their entirety.

A sequencing technique that can be used in the methods of the provided invention includes, for example, Helicos True Single Molecule Sequencing (tSMS) (Harris T. D. et al. (2008) Science 320:106-109), the contents of which are incorporated by reference herein in their entirety. Further description of tSMS is shown, for example, in Lapidus et al. (U.S. Pat. No. 7,169,560), the contents of which are incorporated by reference herein in their entirety, Lapidus et al. (U.S. patent application publication number 2009/0191565, the contents of which are incorporated by reference herein in their entirety), Quake et al. (U.S. Pat. No. 6,818,395, the contents of which are incorporated by reference herein in their entirety), Harris (U.S. Pat. No. 7,282,337, the contents of which are incorporated by reference herein in their entirety), Quake et al. (U.S. patent application publication number 2002/0164629, the contents of which are incorporated by reference herein in their entirety), and Braslaysky, et al., PNAS (USA), 100: 3960-3964 (2003), the contents of which are incorporated by reference herein in their entirety.

Another example of a DNA sequencing technique that can be used in the methods of the provided invention is 454 sequencing (Roche) (Margulies, M et al. 2005, Nature, 437, 376-380, the contents of which are incorporated by reference herein in their entirety). Another example of a DNA sequencing technique that can be used in the methods of the provided invention is SOLiD technology (Applied Biosystems). Another example of a DNA sequencing technique that can be used in the methods of the provided invention is Ion Torrent sequencing (U.S. patent application publication numbers 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the contents of each of which are incorporated by reference herein in their entirety).

In some embodiments, the sequencing technology is Illumina sequencing. Illumina sequencing is based on the amplification of DNA on a solid surface using fold-back PCR and anchored primers. Genomic DNA can be fragmented, or in the case of cfDNA, fragmentation is not needed due to the already short fragments. Adapters are ligated to the 5′- and 3′-ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. Yet another example of a sequencing technique that can be used in the methods of the provided invention is nanopore sequencing (Soni G V and Meller A. (2007) Clin Chem 53: 1996-2001, the contents of which are incorporated by reference herein in their entirety). Another example of a sequencing technique that can be used in the methods of the provided invention involves using a chemical-sensitive field effect transistor (chemFET) array to sequence DNA (for example, as described in US Patent Application Publication No. 20090026082, the contents of which are incorporated by reference herein in their entirety). Another example of a sequencing technique that can be used in the methods of the provided invention involves using an electron microscope (Moudrianakis E. N. and Beer M. Proc Natl Acad Sci USA. 1965 March; 53:564-71, the contents of which are incorporated by reference herein in their entirety).

If the nucleic acid from the sample is degraded or only a minimal amount of nucleic acid can be obtained from the sample, PCR can be performed on the nucleic acid in order to obtain a sufficient amount of nucleic acid for sequencing (See, e.g., Mullis et al. U.S. Pat. No. 4,683,195, the contents of which are incorporated by reference herein in its entirety).

### Biological Samples

Aspects of the invention involve obtaining a test sample, e.g., a biological sample, such as a tissue and/or body fluid sample, from a subject for purposes of analyzing a plurality of nucleic acids (e.g., a plurality of RNA molecules) therein. Samples in accordance with embodiments of the invention can be collected in any clinically-acceptable manner. Any test sample suspected of containing a plurality of nucleic acids can be used in conjunction with the methods of the present invention. In some embodiments, a test sample can comprise a tissue, a body fluid, or a combination thereof. In some embodiments, a biological sample is collected from a healthy subject. In some embodiments, a biological sample is collected from a subject who is known to have a particular disease or disorder (e.g., a particular cancer or tumor). In some embodiments, a biological sample is collected from a subject who is suspected of having a particular disease or disorder.

As used herein, the term “tissue” refers to a mass of connected cells and/or extracellular matrix material(s). Non-limiting examples of tissues that are commonly used in conjunction with the present methods include skin, hair, finger nails, endometrial tissue, nasal passage tissue, central nervous system (CNS) tissue, neural tissue, eye tissue, liver tissue, kidney tissue, placental tissue, mammary gland tissue, gastrointestinal tissue, musculoskeletal tissue, genitourinary tissue, bone marrow, and the like, derived from, for example, a human or non-human mammal. Tissue samples in accordance with embodiments of the invention can be prepared and provided in the form of any tissue sample types known in the art, such as, for example and without limitation, formalin-fixed paraffin-embedded (FFPE), fresh, and fresh frozen (FF) tissue samples.

As used herein, the term “body fluid” refers to a liquid material derived from a subject, e.g., a human or non-human mammal. Non-limiting examples of body fluids that are commonly used in conjunction with the present methods include mucous, blood, plasma, serum, serum derivatives, synovial fluid, lymphatic fluid, bile, phlegm, saliva, sweat, tears, sputum, amniotic fluid, menstrual fluid, vaginal fluid, semen, urine, cerebrospinal fluid (CSF), such as lumbar or ventricular CSF, gastric fluid, a liquid sample comprising one or more material(s) derived from a nasal, throat, or buccal swab, a liquid sample comprising one or more materials derived from a lavage procedure, such as a peritoneal, gastric, thoracic, or ductal lavage procedure, and the like.

In some embodiments, a test sample can comprise a fine needle aspirate or biopsied tissue. In some embodiments, a test sample can comprise media containing cells or biological material. In some embodiments, a test sample can comprise a blood clot, for example, a blood clot that has been obtained from whole blood after the serum has been removed. In some embodiments, a test sample can comprise stool. In one preferred embodiment, a test sample is drawn whole blood. In one aspect, only a portion of a whole blood sample is used, such as plasma, red blood cells, white blood cells, and platelets. In some embodiments, a test sample is separated into two or more component parts in conjunction with the present methods. For example, in some embodiments, a whole blood sample is separated into plasma, red blood cell, white blood cell, and platelet components.

In some embodiments, a test sample includes a plurality of nucleic acids not only from the subject from which the test sample was taken, but also from one or more other organisms, such as viral DNA/RNA that is present within the subject at the time of sampling.

Nucleic acid can be extracted from a test sample according to any suitable methods known in the art, and the extracted nucleic acid can be utilized in conjunction with the methods described herein. See, e.g., Maniatis, et al., Molecular Cloning: A Laboratory Manual, Cold Spring Harbor, N.Y., pp. 280-281, 1982, the contents of which are incorporated by reference herein in their entirety.

In one preferred embodiment, cell free nucleic acid (e.g., cell-free DNA (cfDNA) and/or cell-free RNA (cfRNA)) are extracted from a test sample. cfDNA are short base nuclear-derived DNA fragments present in several bodily fluids (e.g. plasma, stool, urine). See, e.g., Mouliere and Rosenfeld, PNAS 112(11): 3178-3179 (March 2015); Jiang et al., PNAS (March 2015); and Mouliere et al., Mol Oncol, 8(5):927-41 (2014). Tumor-derived circulating tumor nucleic acids (e.g., ctDNA and/or ctRNA) constitutes a minority population of cfNAs (i.e., cfDNA and/or cfRNA), in some cases, varying up to about 50%. In some embodiments, ctDNA and/or ctRNA varies depending on tumor stage and tumor type. In some embodiments, ctDNA and/or ctRNA varies from about 0.001% up to about 30%, such as about 0.01% up to about 20%, such as about 0.01% up to about 10%. The covariates of ctDNA and/or ctRNA are not fully understood, but appear to be positively correlated with tumor type, tumor size, and tumor stage. E.g., Bettegowda et al, Sci Trans Med, 2014; Newmann et al, Nat Med, 2014. Despite the challenges associated with the low population of ctDNA/ctRNA in cfNAs, tumor variants have been identified in ctDNA and/or ctRNA across a wide span of cancers. E.g., Bettegowda et al, Sci Trans Med, 2014. Furthermore, analysis of cfDNA and/or cfRNA versus tumor biopsy is less invasive, and methods for analyzing, such as sequencing, enable the identification of sub-clonal heterogeneity. Analysis of cfDNA and/or cfRNA has also been shown to provide for more uniform genome-wide sequencing coverage as compared to tumor tissue biopsies. In some embodiments, a plurality of cfDNA and/or cfRNA are extracted from a sample in a manner that reduces or eliminates co-mingling of cfDNA and genomic DNA. For example, in some embodiments, a sample is processed to isolate a plurality of the cfDNA and/or cfRNA therein in less than about 2 hours, such as less than about 1.5, 1 or 0.5 hours.

A non-limiting example of a procedure for preparing nucleic acid from a blood sample follows. Blood may be collected in 10 mL EDTA tubes (for example, the BD VACUTAINER® family of products from Becton Dickinson, Franklin Lakes, N.J.), or in collection tubes that are adapted for isolation of cfDNA (for example, the CELL FREE DNA BCT® family of products from Streck, Inc., Omaha, Nebr.) can be used to minimize contamination through chemical fixation of nucleated cells, but little contamination from genomic DNA is observed when samples are processed within 2 hours or less, as is the case in some embodiments of the present methods. Beginning with a blood sample, plasma may be extracted by centrifugation, e.g., at 3000 rpm for 10 minutes at room temperature minus brake. Plasma may then be transferred to 1.5 ml tubes in 1 ml aliquots and centrifuged again at 7000 rpm for 10 minutes at room temperature. Supernatants can then be transferred to new 1.5 ml tubes. At this stage, samples can be stored at −80° C. In certain embodiments, samples can be stored at the plasma stage for later processing, as plasma may be more stable than storing extracted cfDNA and/or cfRNA.

Plasma DNA and/or RNA can be extracted using any suitable technique. For example, in some embodiments, plasma DNA and/or RNA can be extracted using one or more commercially available assays, for example, the QIAmp Circulating Nucleic Acid Kit family of products (Qiagen N.V., Venlo Netherlands). In certain embodiments, the following modified elution strategy may be used. DNA and/or RNA may be extracted using, e.g., a QIAmp Circulating Nucleic Acid Kit, following the manufacturer's instructions (maximum amount of plasma allowed per column is 5 mL). If cfDNA and/or cfRNA are being extracted from plasma where the blood was collected in Streck tubes, the reaction time with proteinase K may be doubled from 30 min to 60 min. Preferably, as large a volume as possible should be used (i.e., 5 mL). In various embodiments, a two-step elution may be used to maximize cfDNA and/or cfRNA yield. First, DNA and/or RNA can be eluted using 30 μL of buffer AVE for each column. A minimal amount of buffer necessary to completely cover the membrane can be used in the elution in order to increase cfDNA and/or cfRNA concentration. By decreasing dilution with a small amount of buffer, downstream desiccation of samples can be avoided to prevent melting of double stranded DNA or material loss. Subsequently, about 30 μL of buffer for each column can be eluted. In some embodiments, a second elution may be used to increase DNA and/or RNA yield.

In other embodiments, RNA can be extracted and/or isolated using any suitable technique. For example, in some embodiments, RNA can be extracted using a commercially-available kit and/or protocol, e.g., a QIAamp Circulating Nucleic Acids kit and micro RNA extraction protocol.

In some embodiments, the methods involve DNase treating an extracted nucleic acid sample to remove cell-free DNA from a mixed cfDNA and cfRNA test sample.

### Computer Systems and Devices

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer programs include, by way of example, both general and special purpose microprocessors, and any one or more processors of any kind of digital computer. Generally, a processor will receive instructions and data from a read-only memory or a random access memory, or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including, by way of example, semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through a network by any form or medium of digital data communication, e.g., a communication network. For example, a reference set of data may be stored at a remote location and a computer can communicate across a network to access the reference data set for comparison purposes. In other embodiments, however, a reference data set can be stored locally within the computer, and the computer accesses the reference data set within the CPU for comparison purposes. Examples of communication networks include, but are not limited to, cell networks (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, a data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification (RFID) tags or chips, or any other medium that can be used to store the desired information, and which can be accessed by a computing device.

Functions described herein can be implemented using software, hardware, firmware, hardwiring, or combinations of any of these. Any of the software can be physically located at various positions, including being distributed such that portions of the functions are implemented at different physical locations.

As one skilled in the art would recognize as necessary or best-suited for performance of the methods of the invention, a computer system for implementing some or all of the described inventive methods can include one or more processors (e.g., a central processing unit (CPU) a graphics processing unit (GPU), or both), main memory and static memory, which communicate with each other via a bus.

A processor will generally include a chip, such as a single core or multi-core chip, to provide a central processing unit (CPU). A process may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Preferably, each computer includes a non-transitory memory such as a solid state drive, flash drive, disk drive, hard drive, etc.

While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present invention. These terms shall accordingly be taken to include, but not be limited to, one or more solid-state memories (e.g., subscriber identity module (SIM) card, secure digital card (SD card), micro SD card, or solid-state drive (SSD)), optical and magnetic media, and/or any other tangible storage medium or media.

A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

Additionally, systems of the invention can be provided to include reference data. Any suitable genomic data may be stored for use within the system. Examples include, but are not limited to: comprehensive, multi-dimensional maps of the key genomic changes in major types and subtypes of cancer from The Cancer Genome Atlas (TCGA); a catalog of genomic abnormalities from The International Cancer Genome Consortium (ICGC); a catalog of somatic mutations in cancer from COSMIC; the latest builds of the human genome and other popular model organisms; up-to-date reference SNPs from dbSNP; gold standard indels from the 1000 Genomes Project and the Broad Institute; exome capture kit annotations from Illumina, Agilent, Nimblegen, and Ion Torrent; transcript annotations; small test data for experimenting with pipelines (e.g., for new users).

In some embodiments, data is made available within the context of a database included in a system. Any suitable database structure may be used including relational databases, object-oriented databases, and others. In some embodiments, reference data is stored in a relational database such as a “not-only SQL” (NoSQL) database. In certain embodiments, a graph database is included within systems of the invention. It is also to be understood that the term “database” as used herein is not limited to one single database; rather, multiple databases can be included in a system. For example, a database can include two, three, four, five, six, seven, eight, nine, ten, fifteen, twenty, or more individual databases, including any integer of databases therein, in accordance with embodiments of the invention. For example, one database can contain public reference data, a second database can contain test data from a patient, a third database can contain data from healthy individuals, and a fourth database can contain data from sick individuals with a known condition or disorder. It is to be understood that any other configuration of databases with respect to the data contained therein is also contemplated by the methods described herein.

References and citations to other documents, such as patents, patent applications, patent publications, journals, books, papers, web contents, have been made throughout this disclosure. All such documents are hereby incorporated herein by reference in their entirety for all purposes.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof. All references cited throughout the specification are expressly incorporated by reference herein.

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

While the present invention has been described with reference to the specific embodiments thereof, it should be understood by those skilled in the art that various changes may be made and equivalents may be substituted without departing from the true spirit and scope of the invention. In addition, many modifications may be made to adapt to a particular situation, material, composition of matter, process, process step or steps, to the objective, spirit and scope of the present invention. All such modifications are intended to be within the scope of the claims appended hereto.

### EXAMPLES

### Example 1: Increasing Percentage of Duplex DNA in Sequencing Pools

The efficacy of exonuclease treatment on formation of collapsed reads and duplex DNA enrichment was tested by incorporating T5 exonuclease and exonuclease V (RecBD) into a sequencing library preparation protocol. The standard library preparation protocol was modified by conducting a ligation reaction with stem-loop adapters instead of UMI adapters, and increasing the ligation time. In some experiments, the ligation time was increased by several hours, such as 5 to 18 hours, such as about 6, 7, 8, 9, 10, 11, 12, 13, 14, 15, 16, or 17 hours. In one experiment, the ligation time was increased to 16.5 hours. An exonuclease enzyme (T5 or RecBD) was added to the reaction mixture and incubated for 30 minutes. Standard post-ligation cleanup was conducted using an SPRI protocol. A USER enzyme was added at the PCR step, and NEB adapters were incorporated. Non-limiting examples of NEB adapters that can be used in conjunction with the present methods are those used in the NEBNext® Ultra DNA Library Prep Kit from Illumina. Other adapters could be used in conjunction with the present methods, including stem-loop adapters that comprise one or more UMIs.

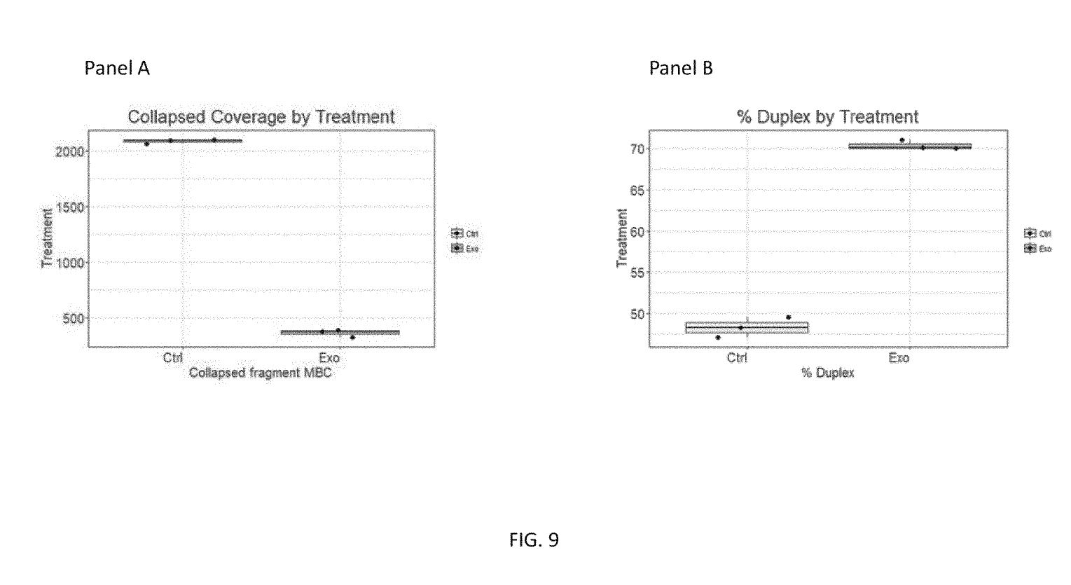

The results are shown in FIG. 9, Panels A and B. As observed previously, overnight ligation with UMI adapters lead to a high amount of dimers and variable library yield (data not shown). In contrast, the incorporation of the exonuclease treatment lead to a reduction in collapsed reads (2100 v. 325) (FIG. 9, Panel A). However, the percentage of duplex DNA increased from approximately 45% to approximately 70% as a result of the inclusion of the exonuclease treatment (FIG. 9, Panel B).

What is claimed is:

1. A method for preparing a sequencing library, the method comprising:
(a) obtaining a test sample comprising a plurality of double-stranded DNA (dsDNA) molecules having first and second ends, wherein the dsDNA molecules comprise a forward strand sequence and a reverse complement strand sequence;
(b) providing a plurality of loop-shaped double-stranded DNA (dsDNA) adapters, wherein the loop-shaped dsDNA adapters comprise a recognition site for nuclease digestion;
(c) modifying the plurality of dsDNA molecules for adapter ligation;
(d) ligating the loop-shaped dsDNA adapters to both ends of the plurality of dsDNA molecules, to generate a plurality of circular adapter-dsDNA-adapter constructs;
(e) amplifying the plurality of circular adapter-dsDNA-adapter constructs to generate a plurality of concatemer amplicons comprising alternating forward and reverse complement strands originating from the dsDNA molecules; and
(f) digesting the plurality of concatemer amplicons to generate a plurality of single-stranded DNA molecules comprising the forward and the reverse complement strand sequences, thereby generating a sequencing library.

2. The method according to claim 1, wherein the loop-shaped dsDNA adapters comprise a unique molecular identifier (UMI).

3. The method according to claim 2, further comprising:
(g) sequencing at least a portion of the sequencing library to obtain a plurality of sequence reads;
(h) grouping the sequence reads into families based on the UMIs, wherein the families comprise a first set of forward strand sequences, each having a first UMI, and a second set of reverse complement strand sequences, each having a second UMI, wherein the second UMI sequence is complementary to the first UMI sequence; and
(i) comparing the sequence reads within each family to generate a consensus sequence for each of the families.

4. The method according to claim 3, further comprising:
(j) aligning the one or more consensus sequences to a reference sequence and identifying the one or more consensus sequences as one or more rare variants if the one or more consensus sequences vary from the reference sequence at one or more nucleotide positions.

5. The method according to claim 1, further comprising contacting the circular adapter-dsDNA-adapter constructs with a topoisomerase enzyme.

6. The method according to claim 1, wherein the dsDNA molecules are cell-free DNA (cfDNA) molecules.

7. The method according to claim 6, wherein the cfDNA molecules originate from healthy cells and from cancer cells.

8. The method according to claim 1, wherein the test sample is from whole blood, a blood fraction, plasma, serum, urine, fecal matter, saliva, a tissue biopsy, pleural fluid, pericardial fluid, cerebrospinal fluid (CSF), or peritoneal fluid.

9. The method according to claim 1, wherein modification of the plurality of dsDNA molecules comprises end-repairing and A-tailing prior to the ligation step.

10. The method according to claim 1, wherein the adapters further comprise a sample-specific index sequence.

11. The method according to claim 1, wherein the adapters further comprise a universal priming site.

12. The method according to claim 1, wherein the adapters further comprise one or more sequencing oligonucleotides for use in cluster generation and/or sequencing.

13. The method according to claim 3, wherein the consensus sequence comprises a sequence of nucleotide bases, wherein each base is identified at a given position in the sequence when a specific base is present in a majority of the sequence reads of the family.

14. The method according to claim 3, wherein the consensus sequence comprises a sequence of nucleotide bases, wherein each base is identified at a given position in the sequence when a specific base is present in at least 70% of the sequence reads comprising the family.

15. The method according to claim 3, wherein the consensus sequence comprises a sequence of nucleotide bases, wherein each base is identified at a given position in the sequence when a specific base is present in at least 80% of the sequence reads comprising the family.

16. The method according to claim 3, wherein the consensus sequence comprises a sequence of nucleotide bases, wherein each base is identified at a given position in the sequence when a specific base is present in at least 90% of the sequence reads comprising the family.

17. The method according to claim 3, wherein the consensus sequence comprises a sequence of nucleotide bases, wherein each base is identified at a given position in the sequence when a specific base is present in at least 95% of the sequence reads comprising the family.

18. The method according to claim 3, further comprising loading at least a portion of the sequence library into a sequencing flow cell and generating a plurality of sequencing clusters on the flow cell, wherein the clusters comprise the forward strand sequence and the reverse complement strand sequence.

19. The method according to claim 3, wherein the sequence reads are obtained from next-generation sequencing (NGS).

20. The method according to claim 3, wherein the sequence reads are obtained from massively parallel sequencing using sequencing-by-synthesis.

21. The method according to claim 3, wherein the sequence reads are obtained from paired-end sequencing.

22. The method according to claim 21, wherein the sequence reads comprise a read pair, wherein each read pair comprises a first read of the forward strand sequence and second read of the reverse complement strand sequence.

23. The method according to claim 4, further comprising using the one or more rare variants to detect the presence or absence of cancer, determine cancer status, monitor cancer progression, and/or determine a cancer classification.

24. The method according to claim 23, wherein monitoring cancer progression further comprises monitoring disease progression, monitoring therapy, or monitoring cancer growth.

25. The method according to claim 23, wherein determining the cancer classification further comprises determining a cancer type and/or a cancer tissue of origin.

26. The method according to claim 23, wherein the cancer comprises a carcinoma, a sarcoma, a myeloma, a leukemia, a lymphoma, a blastoma, a germ cell tumor, or any combination thereof.

27. A method for preparing a sequencing library, the method comprising:
(a) obtaining a test sample comprising a plurality of double-stranded DNA (dsDNA) molecules having first and second ends, wherein the dsDNA molecules comprise a forward strand sequence and a reverse complement strand sequence;
(b) providing a plurality of loop-shaped double-stranded DNA (dsDNA) adapters, wherein the loop-shaped dsDNA adapters comprise a recognition site for nuclease digestion;
(c) modifying the plurality of dsDNA molecules for adapter ligation;
(d) ligating the loop-shaped dsDNA adapters to both ends of the plurality of dsDNA molecules, to generate a plurality of circular adapter-dsDNA-adapter constructs;
(e) digesting unligated nucleic acids with an exonuclease;
(f) cleaving the plurality of loop-shaped dsDNA adapters at the recognition site with a nuclease to generate a sequencing library.

28. The method according to claim 27, wherein the loop-shaped dsDNA adapters comprise a unique molecular identifier (UMI).

29. The method according to claim 28, further comprising:
(g) sequencing at least a portion of the sequencing library to obtain a plurality of sequence reads;
(h) grouping the sequence reads into families based on the UMIs, wherein the families comprise a first set of forward strand sequences, each having a first UMI, and a second set of reverse complement strand sequences, each having a second UMI, wherein the second UMI sequence is complementary to the first UMI sequence; and
(i) comparing the sequence reads within each family to generate a consensus sequence for each of the families.

30. The method according to claim 29, further comprising:
(j) aligning the one or more consensus sequences to a reference sequence and identifying the one or more consensus sequences as one or more rare variants if the one or more consensus sequences vary from the reference sequence at one or more nucleotide positions.

31. The method according to claim 27, further comprising contacting the circular adapter-dsDNA-adapter constructs with a topoisomerase enzyme.

32. The method according to claim 27, wherein the dsDNA molecules are cell-free DNA (cfDNA) molecules.

33. The method according to claim 32, wherein the cfDNA molecules originate from healthy cells and from cancer cells.

34. The method according to claim 27, wherein the test sample is from whole blood, a blood fraction, plasma, serum, urine, fecal matter, saliva, a tissue biopsy, pleural fluid, pericardial fluid, cerebrospinal fluid (CSF), or peritoneal fluid

35. The method according to claim 27, wherein modification of the plurality of dsDNA molecules comprises end-repairing and A-tailing prior to the ligation step.

36. The method according to claim 27, wherein the adapters further comprise a sample-specific index sequence.

37. The method according to claim 27, wherein the adapters further comprise a universal priming site.

38. The method according to claim 27, wherein the adapters further comprise one or more sequencing oligonucleotides for use in cluster generation and/or sequencing.

39. The method according to claim 29, wherein the consensus sequence comprises a sequence of nucleotide bases, wherein each base is identified at a given position in the sequence when a specific base is present in a majority of the sequence reads of the family.

40. The method according to claim 29, wherein the consensus sequence comprises a sequence of nucleotide bases, wherein each base is identified at a given position in the sequence when a specific base is present in at least 70% of the sequence reads comprising the family.

41. The method according to claim 29, wherein the consensus sequence comprises a sequence of nucleotide bases, wherein each base is identified at a given position in the sequence when a specific base is present in at least 80% of the sequence reads comprising the family.

42. The method according to claim 29, wherein the consensus sequence comprises a sequence of nucleotide bases, wherein each base is identified at a given position in the sequence when a specific base is present in at least 90% of the sequence reads comprising the family.

43. The method according to claim 29, wherein the consensus sequence comprises a sequence of nucleotide bases, wherein each base is identified at a given position in the sequence when a specific base is present in at least 95% of the sequence reads comprising the family.

44. The method according to claim 29, further comprising loading at least a portion of the sequence library into a sequencing flow cell and generating a plurality of sequencing clusters on the flow cell, wherein the clusters comprise the forward strand sequence and the reverse complement strand sequence.

45. The method according to claim 29, wherein the sequence reads are obtained from next-generation sequencing (NGS).

46. The method according to claim 29, wherein the sequence reads are obtained from massively parallel sequencing using sequencing-by-synthesis.

47. The method according to claim 29, wherein the sequence reads are obtained from paired-end sequencing.

48. The method according to claim 47, wherein the sequence reads comprise a read pair, wherein each read pair comprises a first read of the forward strand sequence and second read of the reverse complement strand sequence.

49. The method according to claim 30, further comprising using the one or more rare variants to detect the presence or absence of cancer, determine cancer status, monitor cancer progression, and/or determine a cancer classification.

50. The method according to claim 49, wherein monitoring cancer progression further comprises monitoring disease progression, monitoring therapy, or monitoring cancer growth.

51. The method according to claim 49, wherein determining the cancer classification further comprises determining a cancer type and/or a cancer tissue of origin.

52. The method according to claim 49, wherein the cancer comprises a carcinoma, a sarcoma, a myeloma, a leukemia, a lymphoma, a blastoma, a germ cell tumor, or any combination thereof.

53. A method for preparing a sequencing library, the method comprising:
(a) obtaining a test sample comprising a plurality of double-stranded DNA (dsDNA) molecules having first and second ends, wherein the dsDNA molecules comprise a forward strand sequence and a reverse complement strand sequence;
(b) providing a plurality of loop-shaped double-stranded DNA (dsDNA) adapters, wherein the loop-shaped dsDNA adapters comprise a recognition site for nuclease digestion;
(c) modifying the plurality of dsDNA molecules for adapter ligation;
(d) ligating the loop-shaped dsDNA adapters to both ends of the plurality of dsDNA molecules, to generate a plurality of circular adapter-dsDNA-adapter constructs;
(e) digesting unligated DNA molecules with an exonuclease;
(f) amplifying the plurality of circular adapter-dsDNA-adapter constructs to generate a plurality of concatemer amplicons comprising alternating forward and reverse complement strands originating from the dsDNA molecules; and
(g) cleaving the plurality of loop-shaped dsDNA adapters at the nuclease recognition site to generate a plurality of single-stranded DNA molecules comprising the forward and the reverse complement strand sequences, thereby generating a sequencing library.

54. The method according to claim 53, wherein the loop-shaped dsDNA adapters comprise a unique molecular identifier (UMI).

55. The method according to claim 54, further comprising:
(h) sequencing at least a portion of the sequencing library to obtain a plurality of sequence reads;
(i) grouping the sequence reads into families based on the UMIs, wherein the families comprise a first set of forward strand sequences, each having a first UMI, and a second set of reverse complement strand sequences, each having a second UMI, wherein the second UMI sequence is complementary to the first UMI sequence; and
(j) comparing the sequence reads within each family to generate a consensus sequence for each of the families.

56. The method according to claim 55, further comprising:
(k) aligning the one or more consensus sequences to a reference sequence and identifying the one or more consensus sequences as one or more rare variants if the one or more consensus sequences vary from the reference sequence at one or more nucleotide positions.

57. The method according to claim 53, further comprising contacting the circular adapter-dsDNA-adapter constructs with a topoisomerase enzyme.

58. The method according to claim 53, wherein the dsDNA molecules are cell-free DNA (cfDNA) molecules.

59. The method according to claim 58, wherein the cfDNA molecules originate from healthy cells and from cancer cells.

60. The method according to claim 53, wherein the test sample is from whole blood, a blood fraction, plasma, serum, urine, fecal matter, saliva, a tissue biopsy, pleural fluid, pericardial fluid, cerebrospinal fluid (CSF), or peritoneal fluid.

61. The method according to claim 53, wherein modification of the plurality of dsDNA molecules comprises end-repairing and A-tailing prior to the ligation step.

62. The method according to claim 53, wherein the adapters further comprise a sample-specific index sequence.

63. The method according to claim 53, wherein the adapters further comprise a universal priming site.

64. The method according to claim 53, wherein the adapters further comprise one or more sequencing oligonucleotides for use in cluster generation and/or sequencing.

65. The method according to claim 53, wherein the consensus sequence comprises a sequence of nucleotide bases, wherein each base is identified at a given position in the sequence when a specific base is present in a majority of the sequence reads of the family.

66. The method according to claim 53, wherein the consensus sequence comprises a sequence of nucleotide bases, wherein each base is identified at a given position in the sequence when a specific base is present in at least 70% of the sequence reads comprising the family.

67. The method according to claim 53, wherein the consensus sequence comprises a sequence of nucleotide bases, wherein each base is identified at a given position in the sequence when a specific base is present in at least 80% of the sequence reads comprising the family.

68. The method according to claim 53, wherein the consensus sequence comprises a sequence of nucleotide bases, wherein each base is identified at a given position in the sequence when a specific base is present in at least 90% of the sequence reads comprising the family.

69. The method according to claim 53, wherein the consensus sequence comprises a sequence of nucleotide bases, wherein each base is identified at a given position in the sequence when a specific base is present in at least 95% of the sequence reads comprising the family.

70. The method according to claim 53, wherein the method further comprises loading at least a portion of the sequence library into a sequencing flow cell and generating a plurality of sequencing clusters on the flow cell, wherein the clusters comprise the forward strand sequence and the reverse complement strand sequence.

71. The method according to claim 53, wherein the sequence reads are obtained from next-generation sequencing (NGS).

72. The method according to claim 53, wherein the sequence reads are obtained from massively parallel sequencing using sequencing-by-synthesis.

73. The method according to claim 53, wherein the sequence reads are obtained from paired-end sequencing.

74. The method according to claim 73, wherein the sequence reads comprise a read pair, wherein each read pair comprises a first read of the forward strand sequence and second read of the reverse complement strand sequence.

75. The method according to claim 56, further comprising using the one or more rare variants to detect the presence or absence of cancer, determine cancer status, monitor cancer progression, and/or determine a cancer classification.

76. The method according to claim 75, wherein monitoring cancer progression further comprises monitoring disease progression, monitoring therapy, or monitoring cancer growth.

77. The method according to claim 75, wherein determining the cancer classification further comprises determining a cancer type and/or a cancer tissue of origin.

78. The method according to claim 75, wherein the cancer comprises a carcinoma, a sarcoma, a myeloma, a leukemia, a lymphoma, a blastoma, a germ cell tumor, or any combination thereof.
